(19) United States
(12) Patent Application Publication
(10) Pub. No.: US 2022/0023257 A1
(43) Pub. Date: Jan. 27, 2022

(54) TREATMENT OF DEMENTIA-ASSOCIATED TAUOPATHIES

(71) Applicants: Medizinische Hochschule Hannover (MHH), Hannover (DE); Deutsches Zentrum Fur Neurodegenerative Erkrankungen E.V. (DZNE), Bonn (DE)

(72) Inventors: Evgeni Ponimaskin, Hannover (DE); Kian-Fritz Roehrs, Verden/Aller (DE); Malte Butzlaff, Uetze (DE); Josephine Labus, Hannover (DE); Alexander Dityatev, Wahlitz (DE); Hristo Varbanov, Mainz (DE); Jia Shaobo, Magdeburg (DE); Sun Weilun, Magdeburg (DE)

(21) Appl. No.: 17/279,777

(22) PCT Filed: Sep. 30, 2019
(86) PCT No.: PCT/EP2019/076397

§ 371 Date: Mar. 25, 2021

(30) Foreign Application Priority Data
Sep. 28, 2018 (EP) 18197401.5

(51) Int. Cl. A61K 31/40; A61K 31/454; A61K 31/55; A61K 31/5513; A61K 31/496; A61K 31/495; A61K 31/166; A61P 25/28

(52) U.S. Cl. CPC A61K 31/40; A61K 31/454; A61K 31/55; A61P 25/28; A61K 31/496; A61K 31/495; A61K 31/166; A61K 31/5513

(57) ABSTRACT

The present invention relates to a 5-HT7 receptor antagonist for use in preventing or treating a tauopathy. Further, the present invention also relates to a pharmaceutical composition for use in preventing or treating a tauopathy comprising the 5-HT7 receptor antagonist as mentioned above.

### CROSS-REFERENCE TO RELATED APPLICATIONS

This application is the U.S. national phase application filed under 35 U.S.C. § 371 claiming benefit to International Patent Application No. PCT/EP2019/076397, filed on Apr. 2, 2020, which is entitled to priority of EP Application No. 18197401.5, filed Sep. 28, 2018, the disclosures of which are incorporated herein by reference in their entirety.

### REFERENCE TO A “SEQUENCE LISTING,” A TABLE, OR A COMPUTER PROGRAM LISTING APPENDIX SUBMITTED AS AN ASCII TEXT FILE

The present application hereby incorporates by reference the entire contents of the text file named “206119-0047-00US_Sequence_Listing” in ASCII format. The text file containing the Sequence Listing of the present application was created on Mar. 22, 2021 and is 2,125 bytes in size.

### TECHNICAL FIELD OF THE INVENTION

The present invention relates to a 5-HT7 receptor antagonist for use in preventing or treating a tauopathy. Further, the present invention also relates to a pharmaceutical composition for use in preventing or treating a tauopathy comprising the 5-HT7 receptor antagonist as mentioned above.

### BACKGROUND OF THE INVENTION

Aggregation of the microtubule-associated tau protein has been reported in a large portion of neurodegenerative diseases. These so-called tauopathies are characterized by the deposition of hyper-phosphorylated, aggregated tau within the neurons and/or glial cells (Arendt et al., 2016, Brain Res. Bull. 126, 238-292). The most prominent members in this class of disease are Alzheimer's disease (AD) and frontotemporal lobar degeneration (FTLD), causing the majority of dementia illnesses worldwide (Duthey, 2013, Background paper 6.11: Alzheimer disease and other dementias. A public Healthy Approach to Innovation). Intracellular inclusions comprising tau are also found in several other neurodegenerative diseases, including Pick disease, progressive supranuclear palsy, corticobasal degeneration, and frontotemporal dementia with parkinsonism linked to chromosome 17 (FTDP-17) (Josephs, 2017, Mayo Clin. Proc. 92, 1291-1303).

Under physiological conditions, tau participates in regulating microtubule network dynamics by binding and stabilizing microtubules. It also promotes polymerization of tubulin and is known to influence cell morphology, axonal outgrowth and the axonal cargo transport (Wang and Mandelkow, 2015, Nat. Rev. Neurosci. 17, 22-35). Tau's functions are mainly regulated by phosphorylation at multiple sites mediated by a variety of kinases (Buee et al., 2000, Brain Res Brain Res Rev 33, 95-130).

Under pathological conditions, tau phosphorylation is increased, thus resulting in reduced microtubule binding affinity and protein aggregation. In turn, this leads to destabilization of the microtubule network, impairment of axonal transport, inhibition of proteasomal degradation pathways and mitochondrial dysfunction (Arendt et al., 2016, Brain Res. Bull. 126, 238-292).

In recent years, the serotonergic system regained scientific attention as a potential target for the treatment of neurodegenerative diseases associated with increased tau phosphorylation and accumulations of tau proteins.

Since the prevalence of neurodegenerative diseases such as tauopathy is clearly on the rise, and no disease-modifying drugs for the treatment of these diseases are available, the identification of new molecular targets seems to be urgent.

Therefore, there is a need in the art to provide new ways for the prevention and treatment of tauopathy.

Therefore, the objective of the present invention is to comply with this need.

The solution of the present invention is described in the following, exemplified in the appended examples, illustrated in the figures and reflected in the claims.

### SUMMARY OF THE INVENTION

The present invention deals with the target serotonin (5-hydroxytryptamine) receptor 7 (5-HT7R) for treating or preventing tauopaties, such as Alzheimer's disease. It was demonstrated that 5-HT7 receptor antagonists prevent hyperphosphorylation of the tau protein and through that mechanism the formation of tau-tangles in nerve cells is inhibited as well as neurotoxic effects abolished. These findings demonstrate a significant role for 5-HT7R signaling in tau-induced pathology. Following these observations, the application of 5-HT7 receptor antagonists could likely represent an innovative treatment strategy, with potentially disease modifying properties by reducing tau aggregation and subsequent neuronal cell death. Thus, targeting the 5-HT7R reduces abnormal aggregation of tau protein and resulting neurotoxicity, as well as restores neuronal functioning. Therefore, it represents a new promising therapeutic strategy in tau-mediated neurological disorders such as Alzheimer's disease.

In particular, it was demonstrated that 5-HT7 receptor antagonists that target and block 5-HT7R may be used for the prevention or treatment of diseases associated with the accumulation of hyperphosphorylated tau protein (p-tau), such as tauopathies.

The present invention provides evidence that targeting the 5-HT7R may reduce abnormal aggregation of tau protein and resulting neurotoxicity, as well as restore neuronal functioning, thus representing a new promising therapeutic strategy in tau-mediated neurological disorders.

Thus, the present invention relates to a 5-HT7 receptor antagonist for use in preventing or treating a tauopathy.

Additionally, the present invention may comprise the 5-HT7 receptor antagonist for use as mentioned above, wherein the 5-HT7 receptor antagonist preferably has a structure according to the following formula (I),

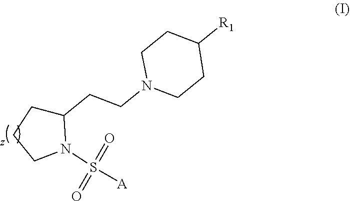

wherein in formula (I)
R₁ is —(C₁-C₆)alkyl, preferably methyl, (C₆-C₁₀)aryl, —O(C₆-C₁₀)aryl, or (C₅-C₁₀)heteroaryl,
which are optionally substituted with one or more substituents independently selected from the group consisting of: halogen, preferably —Cl, or (C₁-C₆)Alkyl;
A is (C₆-C₁₀)aryl, or (C₅-C₁₀)heteroaryl, which are optionally substituted with one or more substituents independently selected from the group consisting of: halogen, —OH, (C₁-C₆)alkyl, or —O(C₁-C₆)alkyl, preferably the at least one substituent is a meta-substituent;
z is 1 or;
a structure according to formula (II)

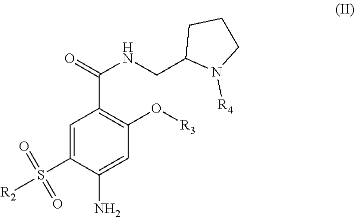

wherein in formula (II)
R₂ and R₃ are independently selected from hydrogen, (C₁-C₆)alkyl, preferably methyl or ethyl, most preferably ethyl;
R₄ is hydrogen, (C₁-C₆)alkyl, preferably methyl or ethyl, most preferably ethyl, or (C₂-C₆)alkenyl;
or a structure according to formula (III)

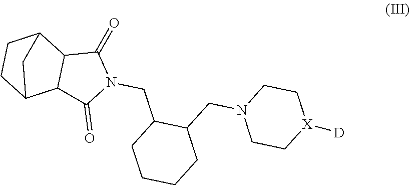

wherein in formula (III)

### X is N or CH;

D is hydrogen, (C₅-C₁₀)heteroaryl, preferably selected from

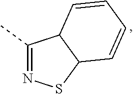

(C₆-C₁₀)aryl, —O(C₆-C₁₀)aryl, or —S(C₆-C₁₀)aryl,
which are optionally substituted with one or more substituents independently selected from the group consisting of: halogen, (C₁-C₆)alkyl, or —OH;
or a structure according to formula (IV)

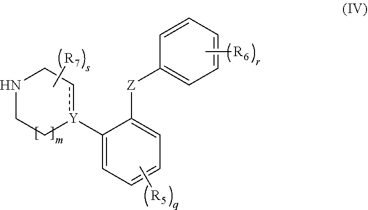

wherein in formula (IV)

### Z is O or S;

### Y is N, C or CH;

s is an integer in the range 0 to 8;
q is an integer in the range 0 to 4;
r is an integer in the range 0 to 5;
m is 1 or 2;
The dotted line represents an optional bond;
R₇ is hydrogen, —(C₁-C₆)alkyl, or two R₇ attached to the same carbon atom may form a 3 to 6 membered spiro attached cyclo-alkyl;
R₅ is hydrogen, halogen, cyano, —NO₂, (C₁-C₆)alkenyl, (C₁-C₆)alkyl, —O(C₁-C₆)alkyl, —OH, hydroxy(C₁-C₆)alkyl, halo(C₁-C₆)alkyl, (C₃-C₈)cycloalkyl, C₃-C₈)cycloalkyl-(C₁-C₆)alkyl, acyl, —CO₂(C₁-C₆)alkyl, —(C₆-C₁₀)aryl, —SO₂(C₁-C₆)alkyl, or —NRₓR_{y};
R₆ is hydrogen, halogen, —CN, —NO₂, (C₁-C₆)alkenyl, (C₁-C₆)alkyl, —O(C₁-C₆)alkyl, —OH, hydroxy(C₁-C₆)alkyl, halo(C₁-C₆)alkyl, (C₃-C₈)cycloalkyl, C₃-C₈)cycloalkyl-(C₁-C₆)alkyl, acyl, —CO₂(C₁-C₆)alkyl, (C₆-C₁₀)aryl, —SO₂(C₁-C₆)alkyl, —NRₓR_{y}; —NRₓCO(C₁-C₆)alkyl, or —CONRₓR_{y};
wherein each Rₓ and R_{y} is independently selected from hydrogen, (C₁-C₆)alkyl, (C₃-C₈)cycloalkyl, (C₃-C₈)cycloalkyl-(C₁-C₆)alkyl, or (C₆-C₁₀)aryl;
or Rₓ and R_{y}, together with the nitrogen to which they are attached from a 3 to 7 membered ring which optionally contains one further heteroatom;
or a structure according to formula (V)

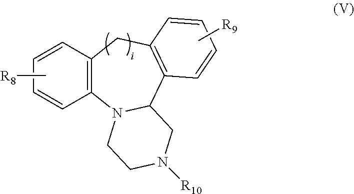

wherein in formula (V)
R₈ and R₉ are independently selected from hydrogen, halogen, —OH, —OCO(C₁-C₆)alkyl, (C₁-C₆)alkyl, —O(C₁-C₆)alkyl, or —CF₃;
R₁₀ is hydrogen, (C₁-C₆)alkyl, or —(C₁-C₆)alkylaryl;
i is 0 or 1;
or a structure according to formula (VI)

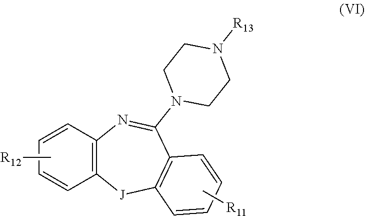

wherein in formula (VI)
R₁₁ and R₁₂ are independently selected from hydrogen, halogen, —OH, (C₁-C₆)alkyl, —CF₃, —O(C₁-C₆)alkyl, —NO₂, —NRₒR_{z}, —SO₂NRₒR_{z}, or —S(C₁-C₆)alkyl;
wherein each Rₒ and R_{z} is independently selected from hydrogen, (C₁-C₆)alkyl;
R₁₃ is hydrogen, allyl, (C₁-C₆)alkyl, preferably (C₁-C₃)alkyl, —O(C₁-C₆)alkyl, or hydroxy(C₁-C₆)alkyl, preferably hydroxy(C₁-C₃)alkyl;

### J is S or NH;

or is a pharmaceutically acceptable salt, prodrug, enantiomer, diastereomer, racemic mixture, crystalline form, amorphous, unsolved form or solvate of the general formula (I), (II), (III), (IV), (V) or (VI).

More preferably, the 5-HT7 receptor antagonist is selected from the group consisting of SB-269970, Amisulpride, Lurasidone, Vortioxetine, Mianserin, Clozapine.

Most preferably, the 5-HT7 receptor antagonist is SB-269970.

Further, the present invention may comprise the 5-HT7 receptor antagonist for use as mentioned above, wherein the antagonist is a small organic molecule comprising at least two carbon atoms having a molecular weight in the range between 100 and 1000 Dalton.

The present invention also comprises a pharmaceutical composition for use in preventing or treating a tauopathy comprising the 5-HT7 receptor antagonist as mentioned above.

Also contemplated by the present invention may be the 5-HT7 receptor antagonist for use as mentioned above, or the pharmaceutical composition as mentioned above, for use in preventing or treating a tauopathy by prevention of both the receptor-induced phosphorylation and accumulation of tau proteins.

Additionally, the present invention may comprise the 5-HT7 receptor antagonist for use as mentioned above, or the pharmaceutical composition as mentioned above, wherein tauopathy is dementia-associated tauopathy.

Further, the present invention may also comprise the 5-HT7 receptor antagonist for use as mentioned above, or the pharmaceutical composition as mentioned above, wherein tauopathy is selected from the group consisting of Alzheimer's disease, frontotemporal dementia, primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), Huntington disease, glial globular tauopathy, amyotrophic lateral sclerosis (ALS), Parkinson's disease, spinal muscular atrophy (SMA), cerebral amyloid angiopathy (CAA).

### BRIEF DESCRIPTION OF THE FIGURES

FIG. 1: 5-HT7R induces tau hyperphosphorylation and accumulation in a Gα12-independent manner.

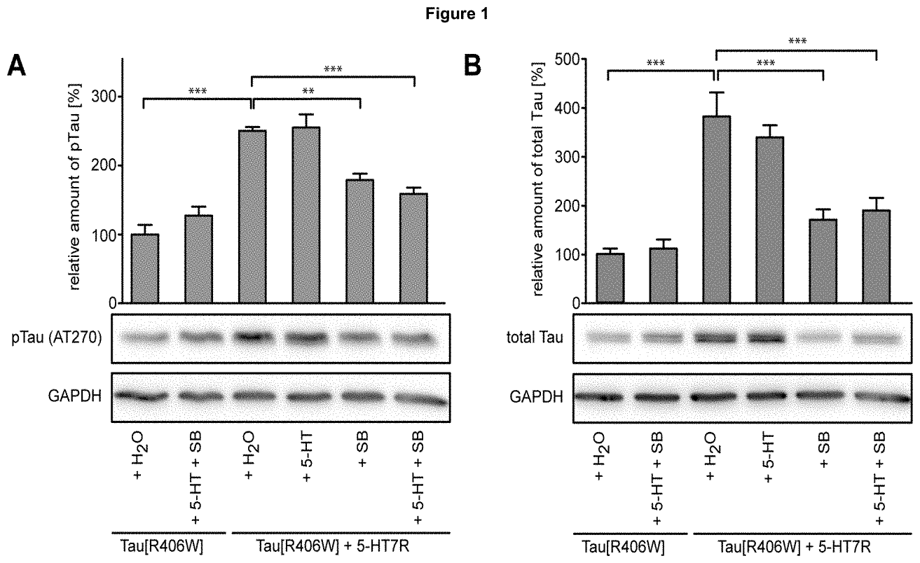

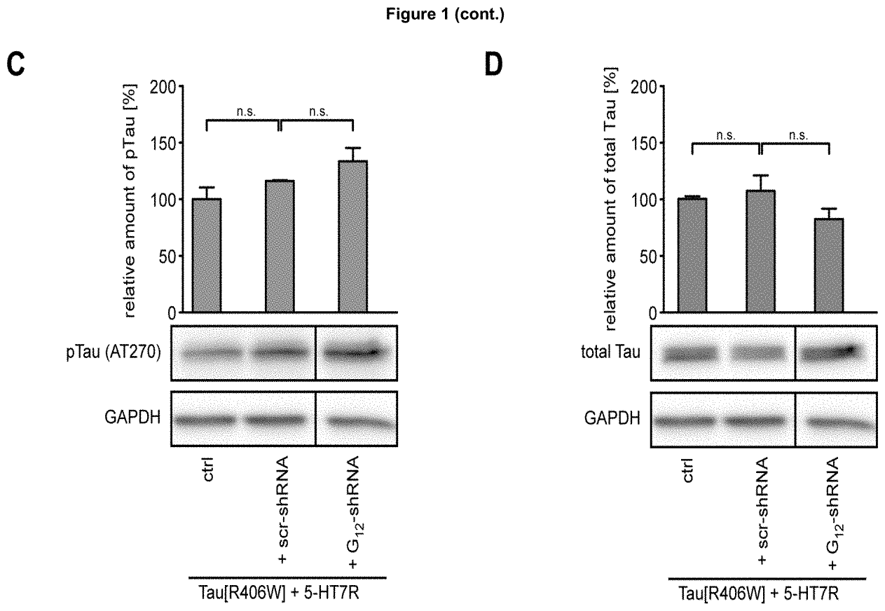

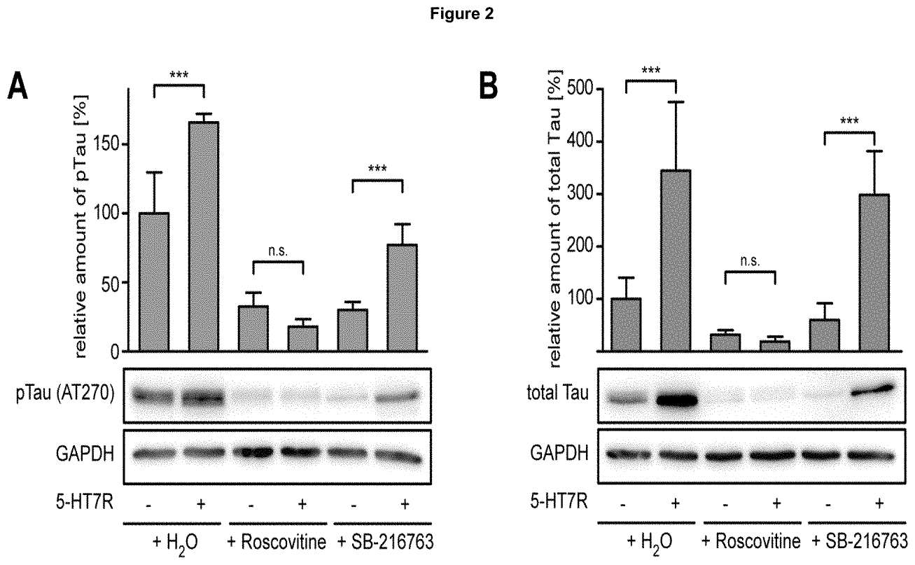

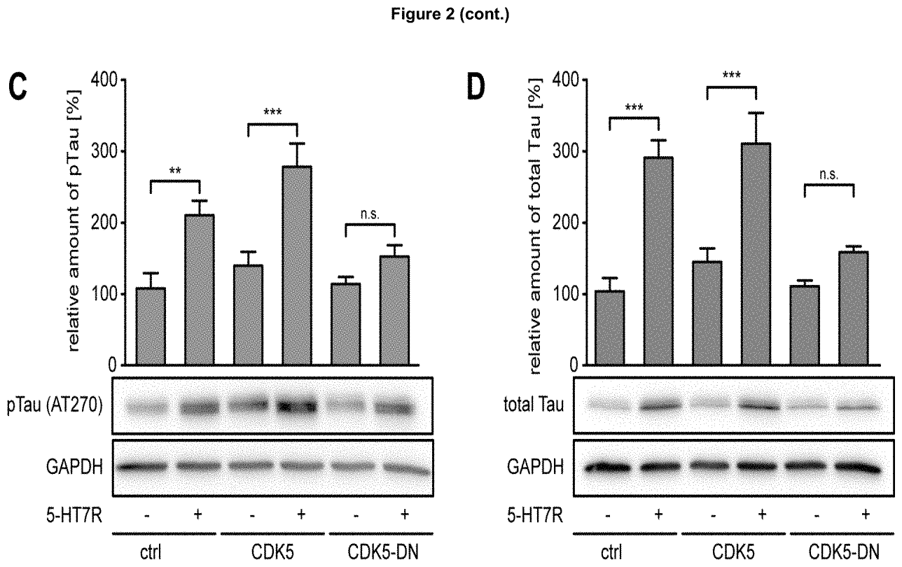

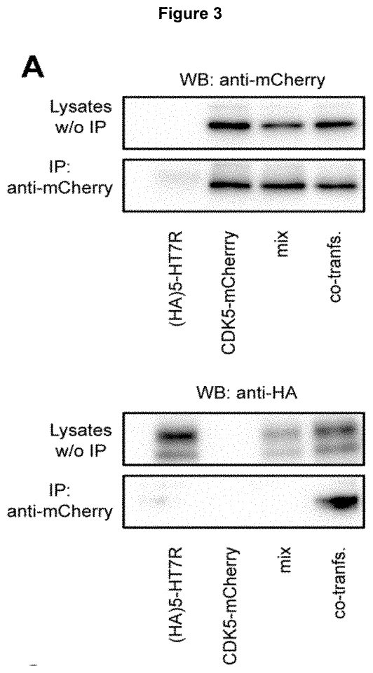

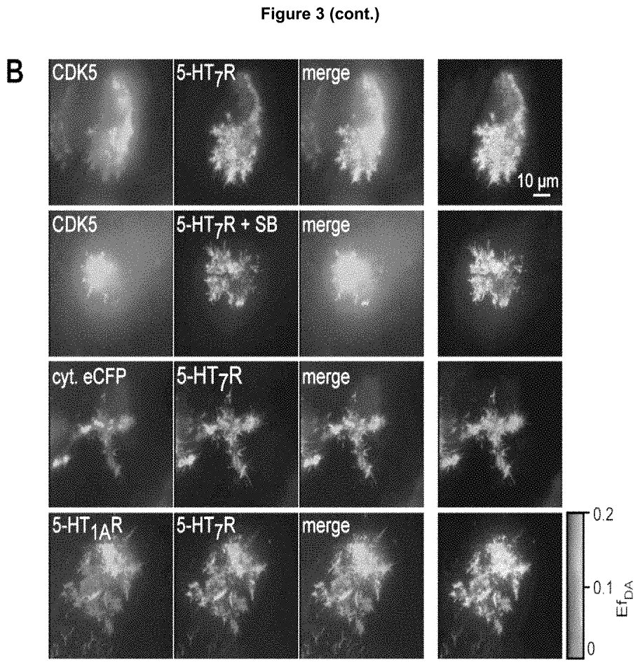

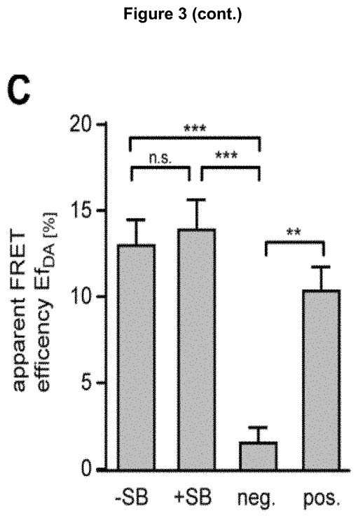

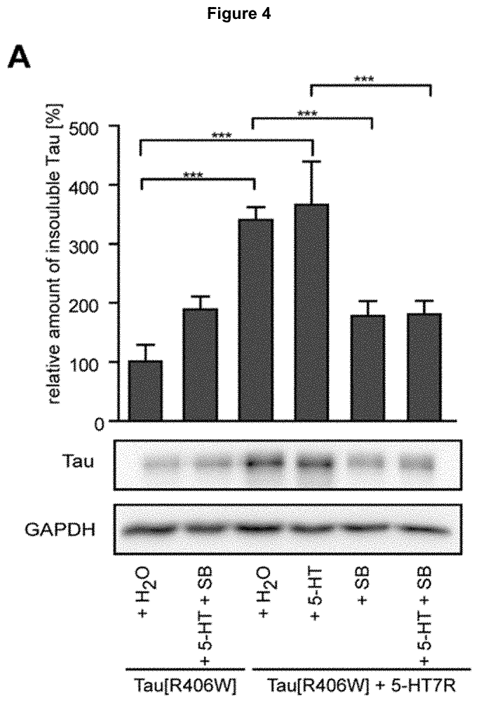

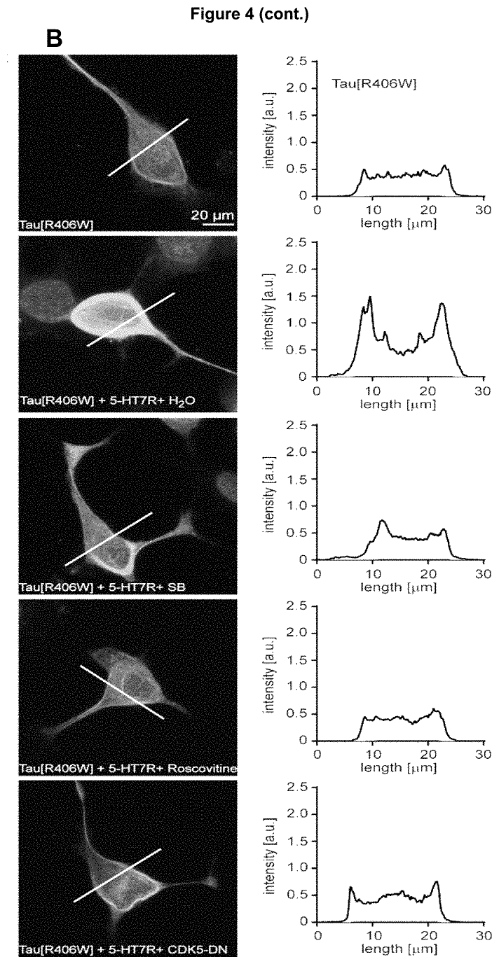

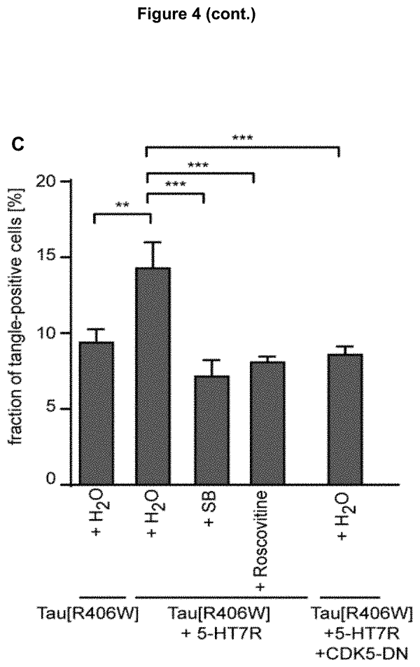

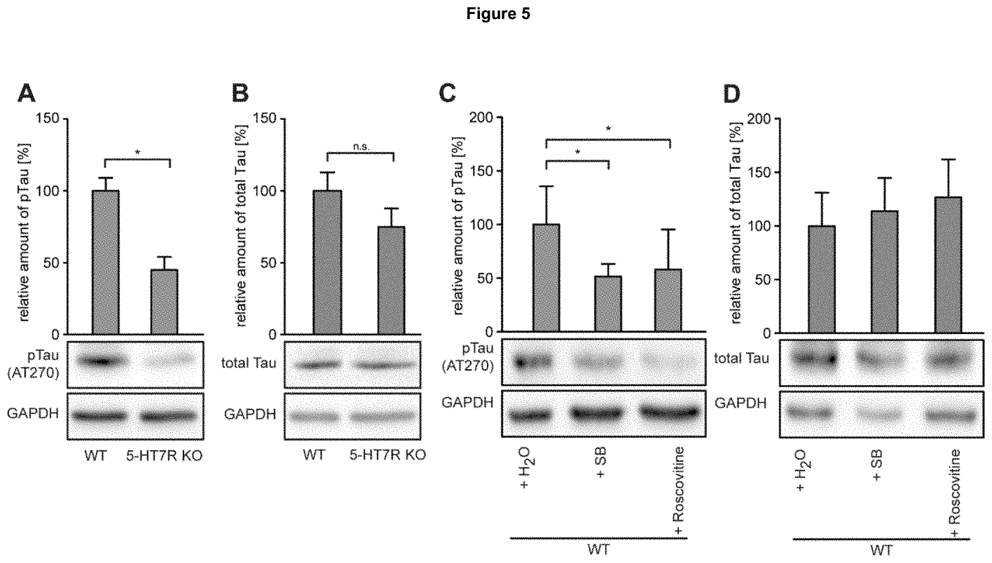

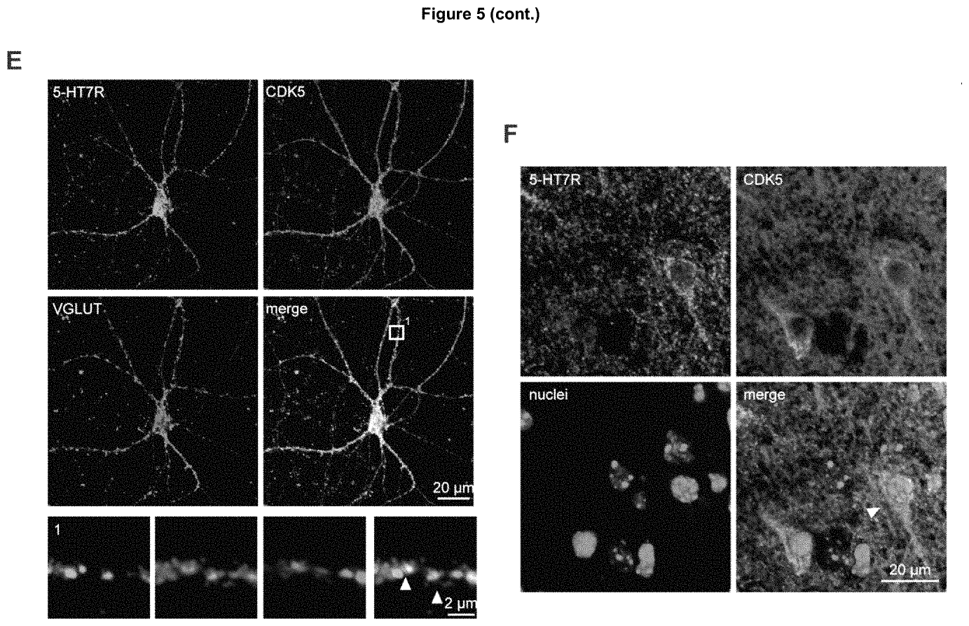

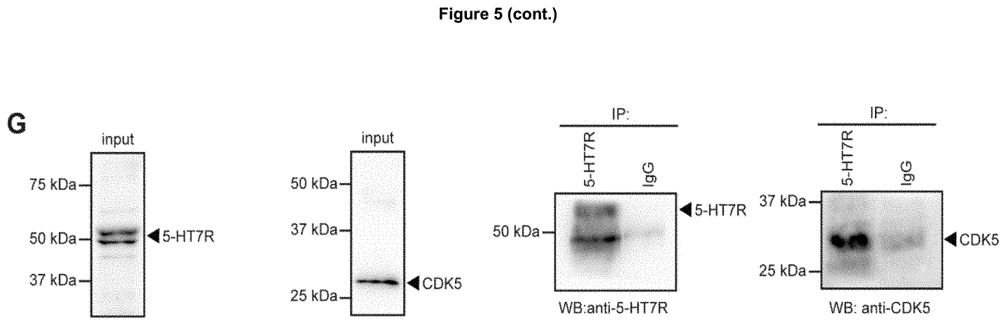

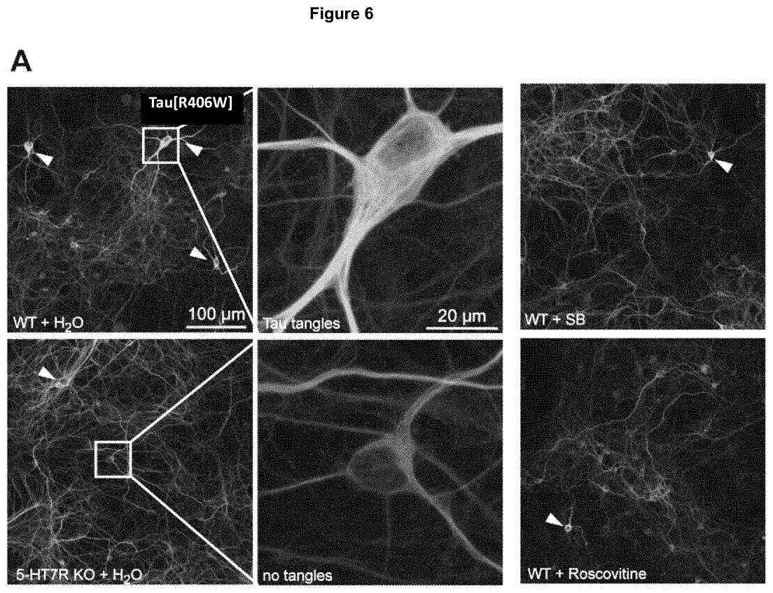

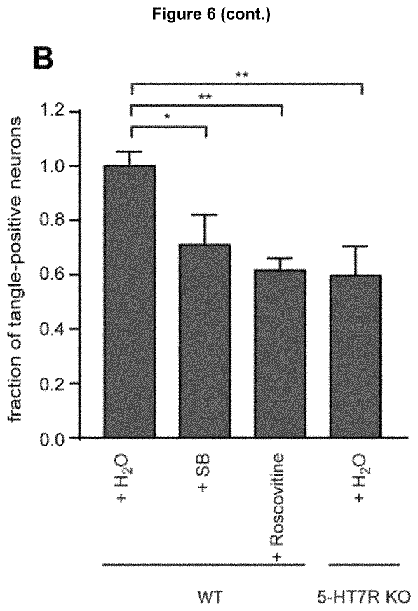

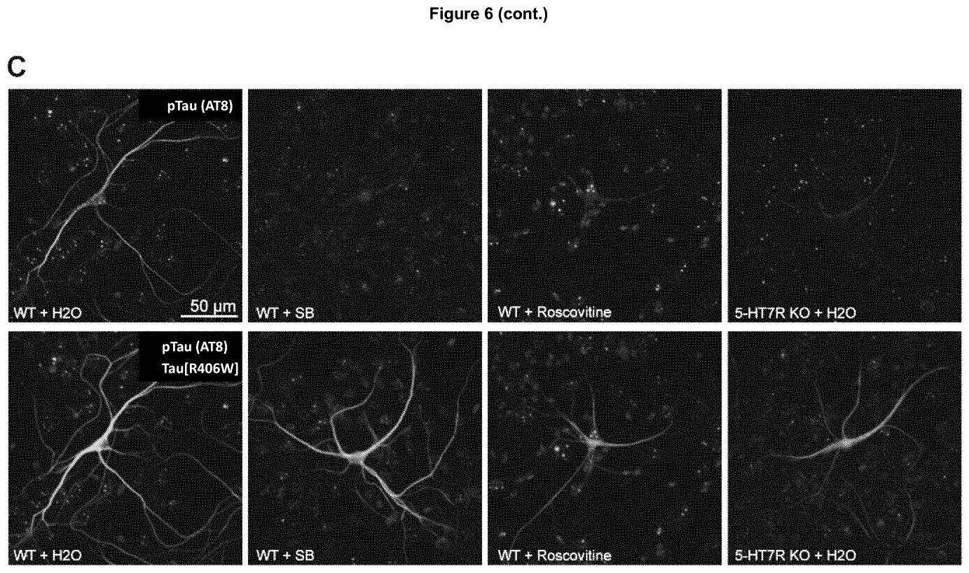

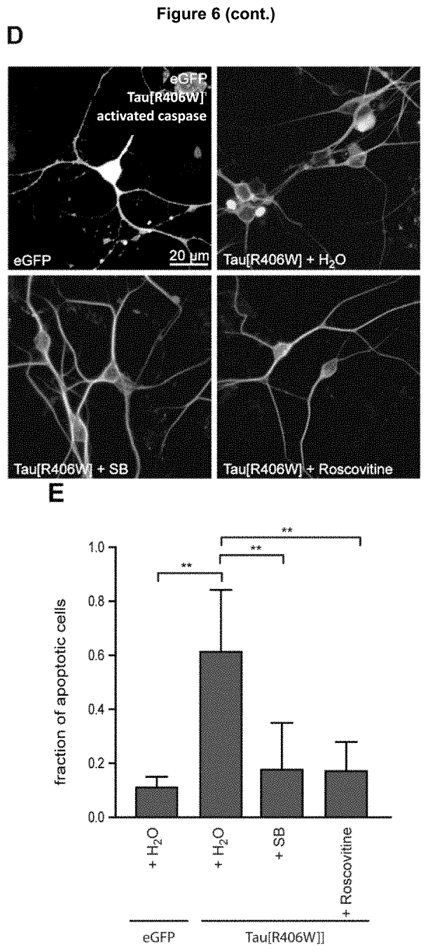

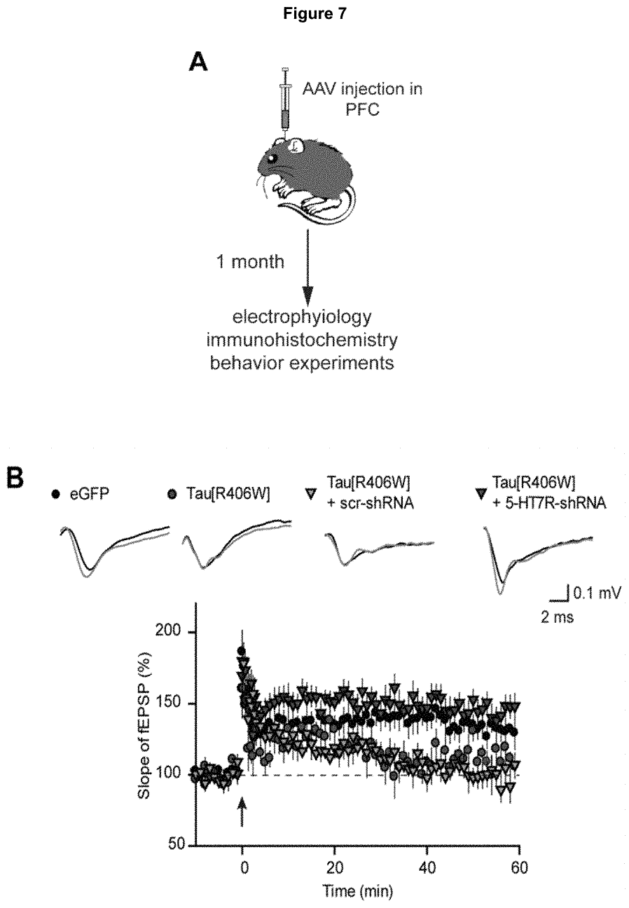

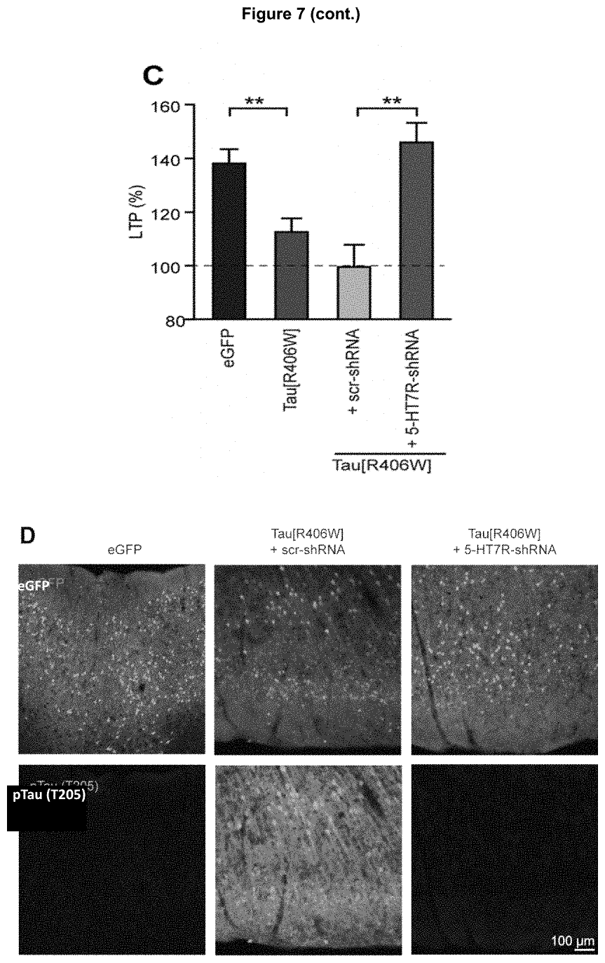

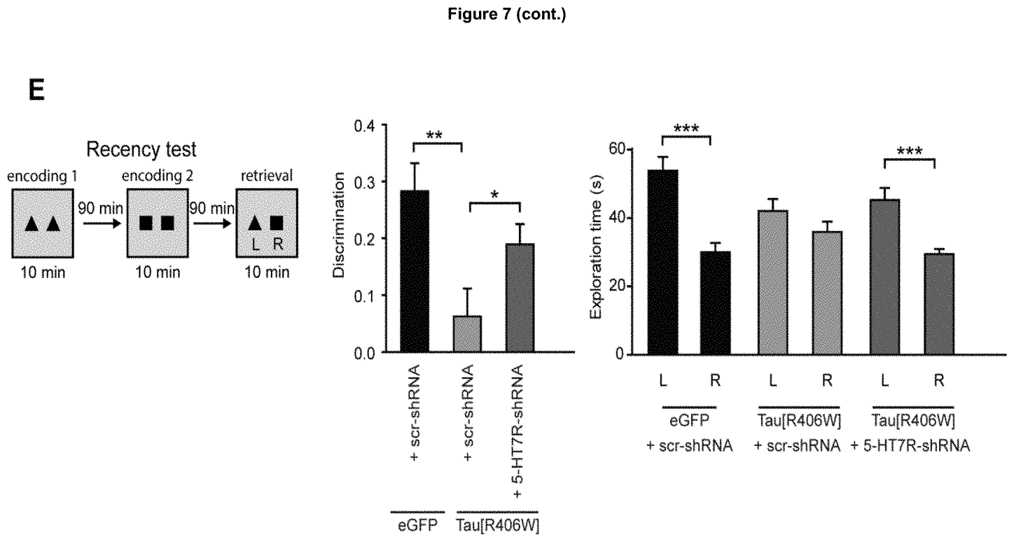

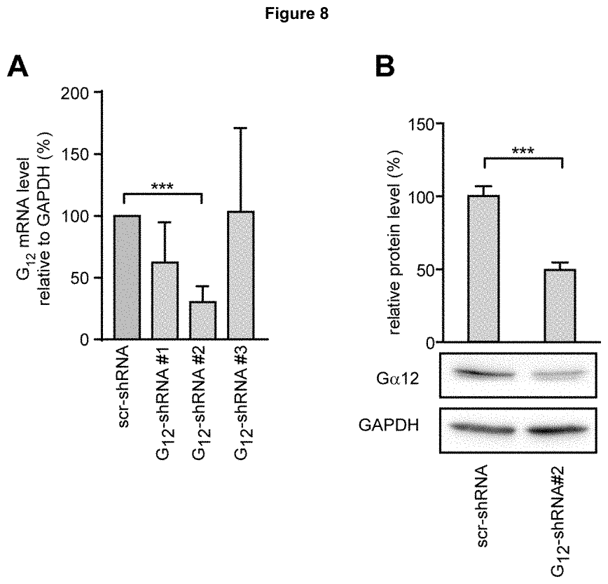

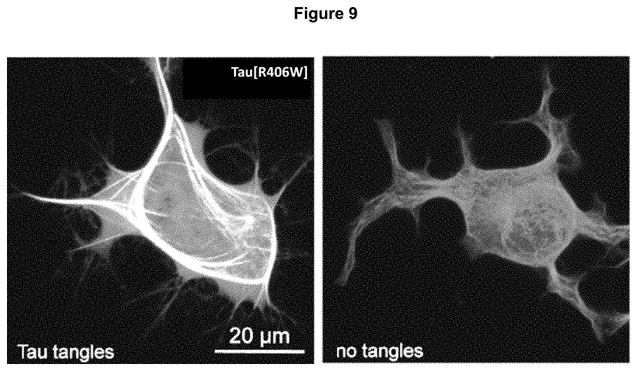

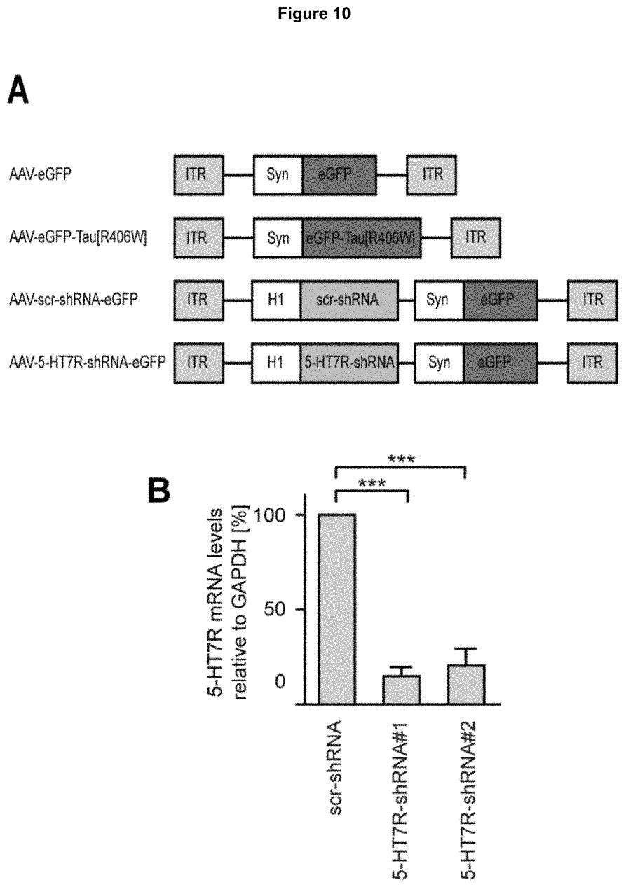

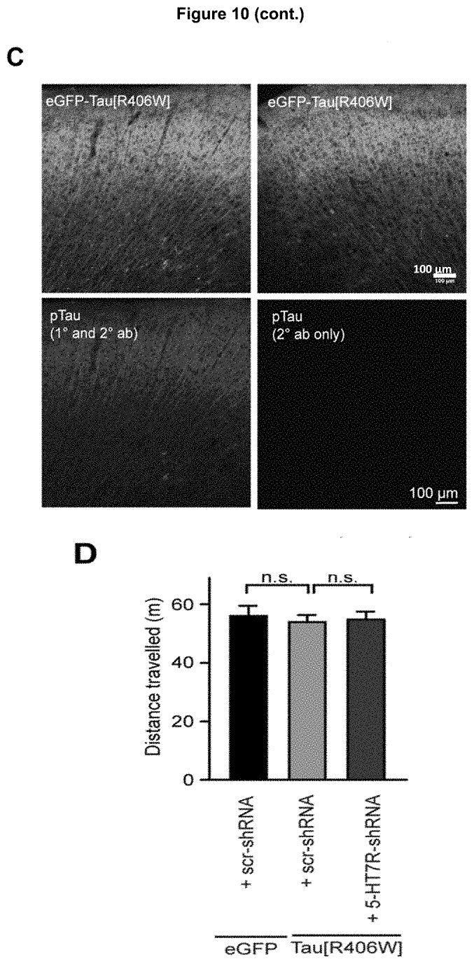

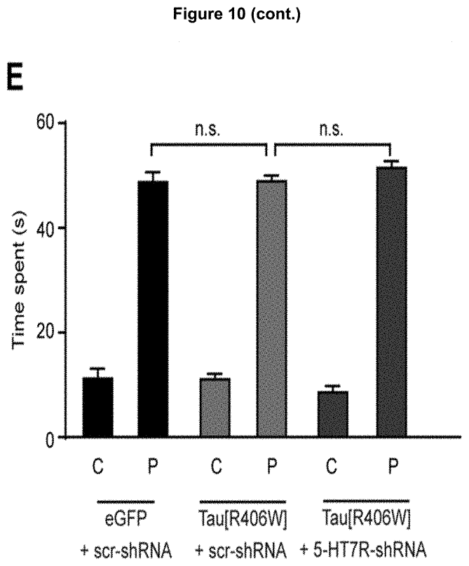

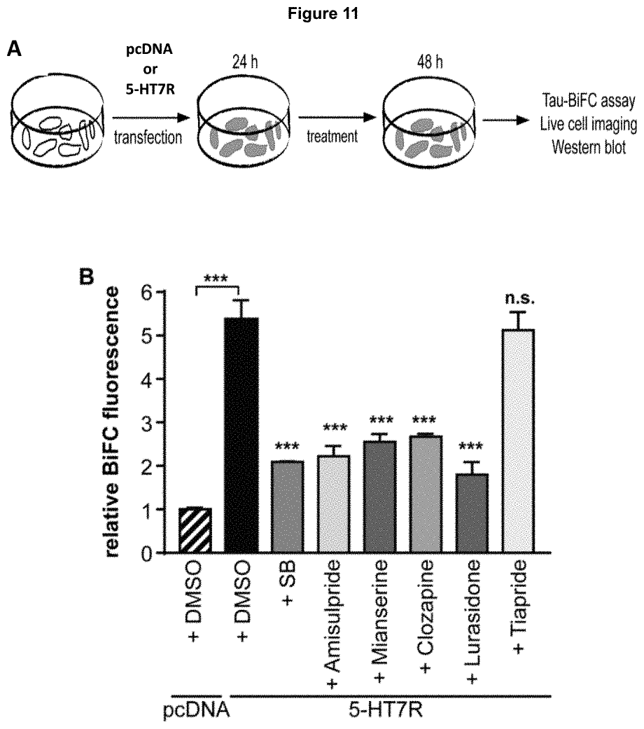

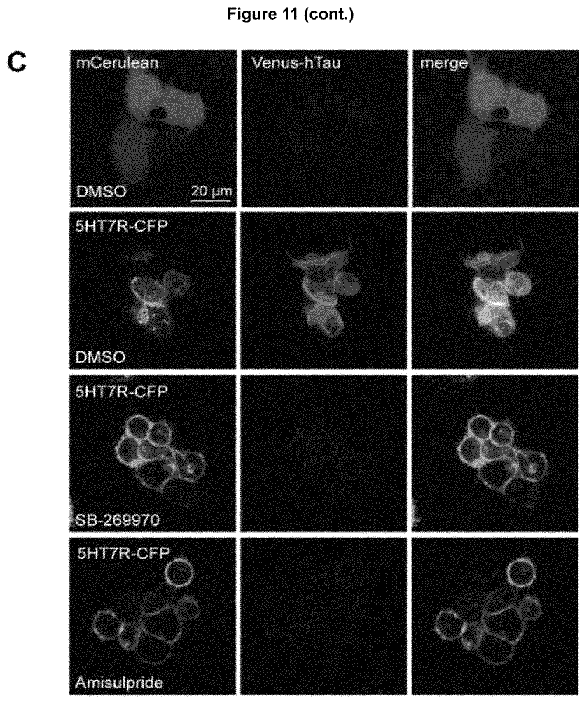

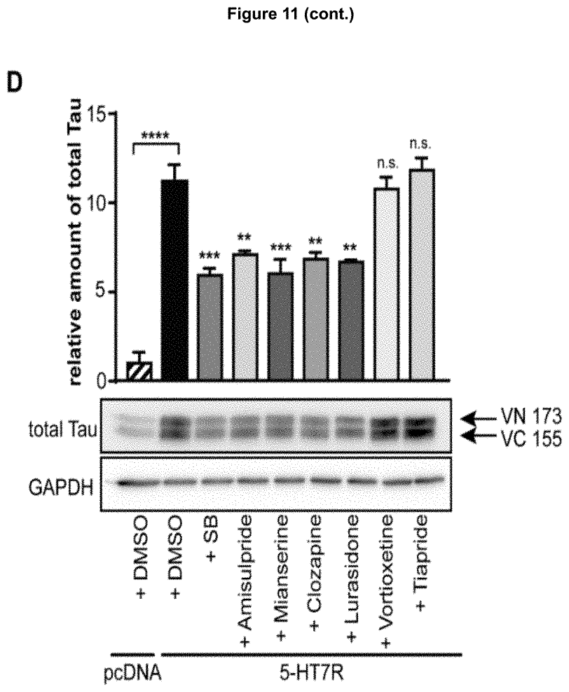

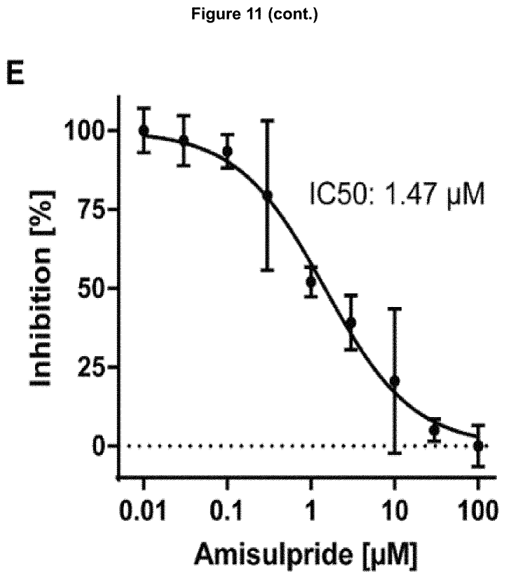

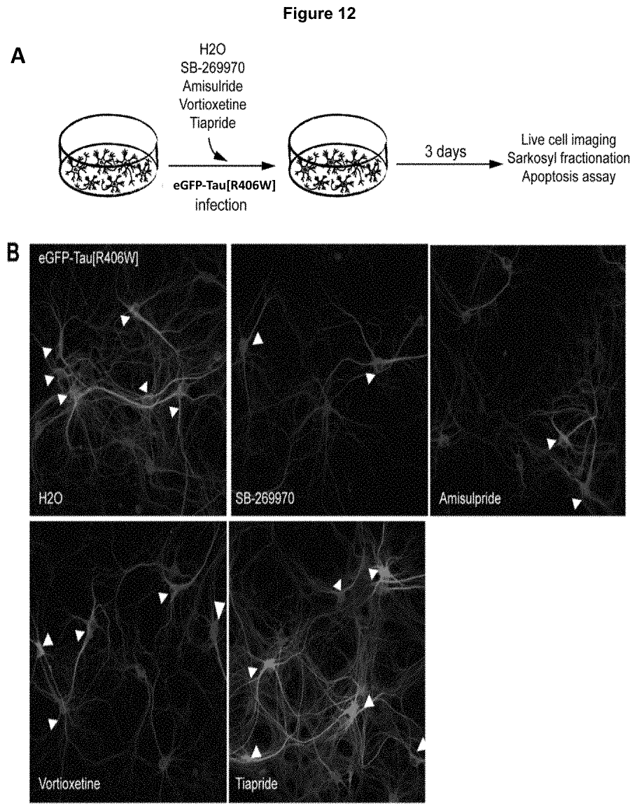

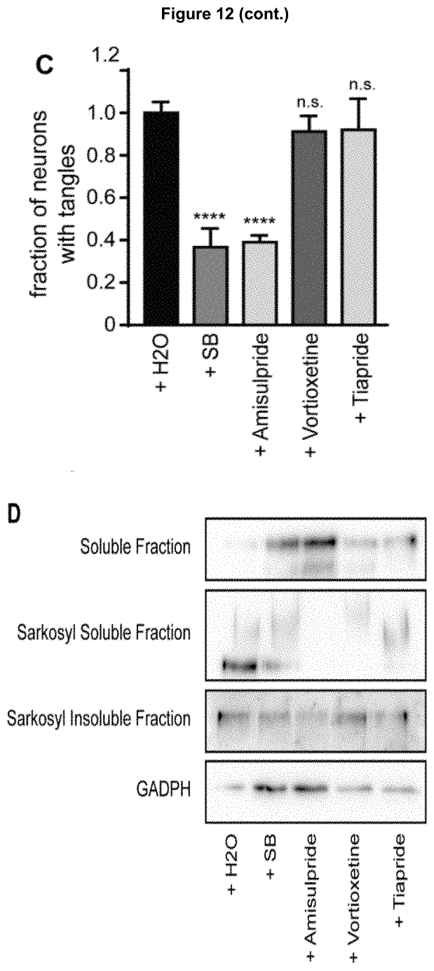

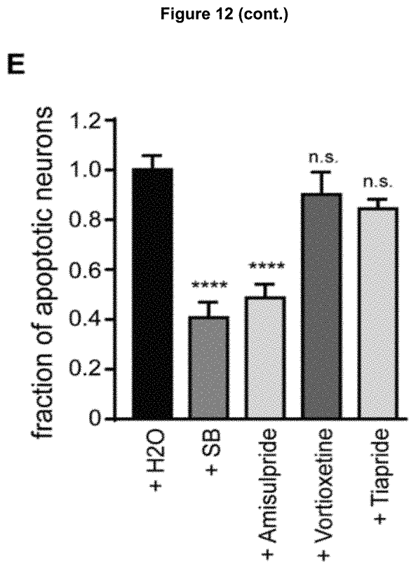

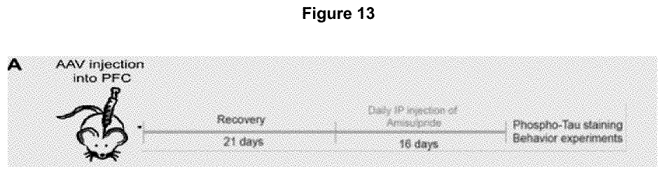

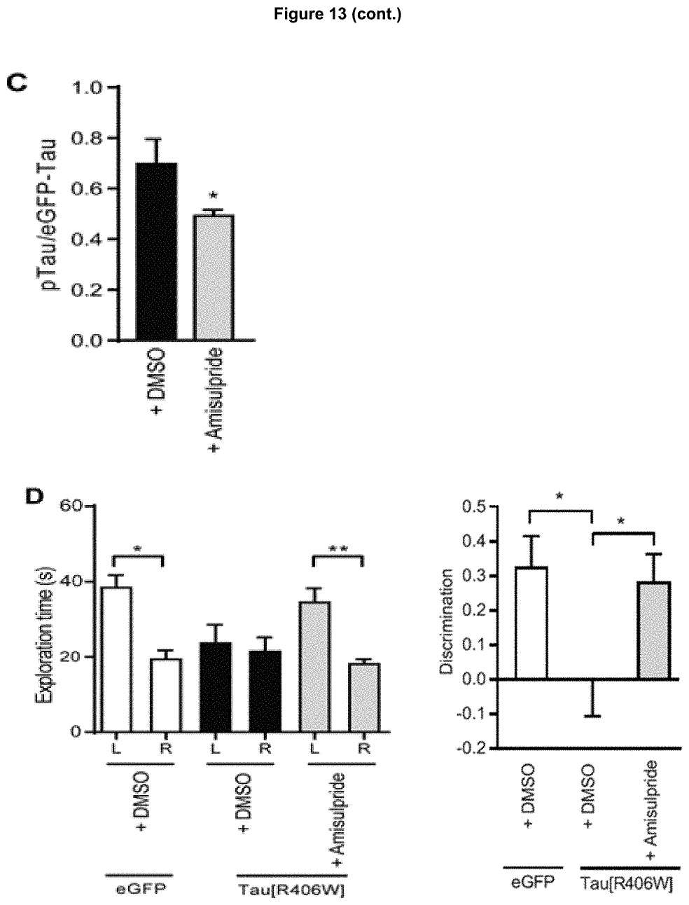

A and B. Representative Western blots and corresponding quantifications are shown. N1E-115 cells were transfected with eGFP-Tau[R406W] mutant alone or together with (HA)5-HT7R, and treated with either H₂O, 20 μM 5-HT, 100 nM SB-269970 alone or in combination with 5-HT. Membranes were probed with phospho-specific tau antibody AT270 (A) or 5A6 antibody (B) for assessing total tau levels. Results were normalized to GAPDH expression and represented as mean+SD. Water-treated cells where set as 100% (N=5). C and D. Representative Western blots and corresponding quantifications are shown. N1E-115 cells were co-transfected with eGFP-Tau[R406W] mutant and (HA)5-HT7R together with either pcDNA (ctrl), scrambled shRNA (scr-shRNA) or specific shRNA directed against the Gα12 subunit of heterotrimeric G-Protein (G₁₂-shRNA, see also FIG. 8). Membranes were probed with phospho-specific tau antibody AT270 (C) or 5A6 (D) for assessing total levels of tau. Results were normalized to GAPDH expression and are represented as mean+SD (N=3). ** indicates statistical significance of p<0.01, *** p<0.001 (one-way ANOVA, followed by Bonferroni's post hoc test).

FIG. 2: 5-HT7R-induced increase in phosphorylation and accumulation of tau is mediated by CDK5, but not GSK3β.

A and B. Representative Western blots and quantifications are shown. N1E-115 cells co-transfected with eGFP-Tau[R406W] mutant and (HA)5-HT7R were treated overnight with 20 μM CDK5 inhibitor Roscovitine or 10 μM GSK3 inhibitor SB-216763. Membranes were probed with phospho-specific tau antibody AT270 (A) or 5A6 (B) for assessing total levels of tau. Results were normalized to GAPDH expression and represented as mean+SD (N=4). C and D. N1E-115 cells were co-transfected with plasmids encoding (HA)5-HT7R and wild-type CDK5-mCherry (CDK5) or dominant negative mutant CDK5-T33-mCherry (CDK5-DN). AT270 antibody was used to detect tau phosphorylation (C) and 5A6 antibody to assess total tau levels (D). Results were normalized to the GAPDH expression and represented as mean+SD (N=6). ** indicates statistical significance of p<0.01, *** p<0.001 (one-way ANOVA, followed by Bonferroni's post hoc test).

FIG. 3: 5-HT7R directly interacts with CDK5.

A. N1E-115 cells overexpressing either (HA)5-HT7R or CDK5-mCherry alone, a mixture of cells individually expressing (HA)5-HT7R or CDK5-mCherry (mix) as well as cells co-expressing (HA)5-HT7R and CDK5-mCherry (co-trans) were subjected to immunoprecipitation (IP) with anti-mCherry antibody followed by Western blotting with either anti-mCherry or anti-HA antibody. Input (lysates w/o IP) was assessed in parallel with the same antibodies. Representative Western blot is shown (N=4). B and C. Representative Total Internal Reflection Fluorescence (TIRF) images and corresponding quantification showing apparent FRET efficiencies Ef_{DA} between CDK5-eCFP and 5-HT7R-eYFP in transfected N1E-115 cells. Cells were treated with (+SB) or without 100 nM SB-269970 (−SB) overnight. Cells co-transfected with cytosolic eCFP and 5-HT7R-eYFP were used as a negative control (neg), while cells co-expressing 5-HT1AR-eCFP and 5-HT7R-eYFP served as positive control (pos; N=3; total analyzed cells 14≤n≤32). ** indicates statistical significance of p<0.01, *** p<0.001 (one-way ANOVA, followed by Bonferroni's post hoc test).

FIG. 4: 5-HT7R induces tau aggregation and tangle formation.

A. Representative Western blot with lysates of N1E-115 cells co-transfected with eGFP-Tau[R406W] mutant and (HA)5-HT7R after sarkosyl fractionation and corresponding quantification. Cells were pre-treated with 100 nM SB-269970 overnight or left untreated, following application of 20 μM 5-HT for 1 h. HRP-conjugated anti-GFP antibody was used to detect total tau levels in the sarkosyl-unsoluble fraction and detected intensities were normalized to GAPDH expression in the soluble fraction. Water-treated and eGFP-Tau[R406W] mutant transfected cells were set 100%. Results are represented as mean+SD (N=3). *** indicates statistical significance of p<0.001 (one-way ANOVA, followed by Bonferroni's post hoc test). B. Quantification of tau tangles in N1E-115 cells. Cells were transfected with the indicated plasmids and treated overnight with 100 nM SB-269970 or 20 μM Roscovitine. The fraction of cells with eGFP-positive filamentous tangles was calculated from all eGFP-positive cells in a confined area in 7 independent experiments (N=7; total analyzed cells 2343≤n≤2799). Experiments were done in a double-blind fashion. See also FIG. 9 showing representative cells with and without tangles. ** indicates statistical significance of p<0.01, *** p<0.001 (one-way ANOVA). C. Representative maximum intensity projections of N1E-115 cells co-transfected with eGFP-Tau[R406W] mutant and (HA)5-HT7R with and without CDK5-DN, treated overnight with 100 nM SB-269970 or 20 μM Roscovitine and stained with phospho-specific tau antibody AT8. Corresponding representative fluorescence intensity profiles display localization of eGFP-Tau[R406W] mutant (eGFP, green) and phosphorylated tau (AT8, red) within the cell.

FIG. 5: Tau phosphorylation in cortical neurons is regulated via 5-HT7R and CDK5.

A and B. Quantifications and representative Western blots of lysates from cultured primary cortical neurons of wild type (WT) or 5-HT7R-KO mice (DIV 4). AT270 antibody was used to detect phosphorylation of tau (A) and tau-5 was used for determination of endogenous total protein levels (B). Results were normalized to GAPDH expression and represented as mean+SD. * indicates statistical significance of p<0.05, n.s. not statistically significant (N=4, student's t-test). C and D. Representative Western blots with lysates of cultured primary cortical neurons of WT mice (DIV 4) after treatment with 100 nM SB-269970, 20 μM Roscovitine or H₂O overnight. AT270 antibody was used to detect phosphorylation of tau (C) and tau-5 was used for determination of endogenous total protein levels (D). Results were normalized to GAPDH expression and represented as mean+SD. * indicates statistical significance of p<0.05 (N=3, one-way ANOVA, followed by Bonferroni's post hoc test). E. Primary cortical neurons of wild type mice (DIV 14) were stained with antibodies against 5-HT7R, CDK5 and VGLUT. White box is magnified in the lower panels. Arrow heads indicate co-localization. F. Cortical slices of P6 wild type mice were stained with antibodies against 5-HT7R and CDK5. Arrow heads indicate co-localization. G. Co-immunoprecipitation experiments for endogenous CDK5 and 5-HT7R. Cortex homogenates were prepared from P6 wild type mice and subjected to immunoprecipitation (IP) with anti-5-HT7R antibody or with rabbit IgG, followed by Western blotting using either anti-5-HT7R or anti-CDK5 antibody. Representative Western blot is shown (N=4).

FIG. 6: Increased tau tangle formation in eGFP-Tau[R406W]-infected primary cortical neurons correlates with increased neuronal apoptosis.

A. Representative images of DIV 14 primary cortical neurons isolated form WT and 5-HT7R-KO mice infected at DIV10 with AAV constructs encoding eGFP-Tau[R406W] and treated with H₂O, 100 nM SB-269970 or 20 μM Roscovitine for 3 days. White boxes and its corresponding magnifications show representative neurons with and without tau tangles. Arrow heads indicate cells that were counted as tangle-positive. B. Quantification of tau tangles. The number of tangle-positive neurons was counted in a confined area in 6 independent experiments and is represented as a fraction of total number of infected neurons. * indicates statistical significance of p<0.05; ** p<0.01 (N=6; total analyzed cells 35511897, one-way ANOVA, followed by Dunnett's post hoc test). C. Representative images of DIV14 primary cortical WT and 5-HT7R-KO neurons infected at DIV 10 with AAV-eGFP-Tau[R406W], treated with 100 nM SB-269970 or 20 μM Roscovitine for 3 days and stained with phospho-specific tau antibody ATB. D. Representative images of DIV14 primary cortical WT neurons infected at DIV10 either with AAV constructs encoding eGFP (white) or eGFP-Tau[R406W] (green). After treatment either with H₂O, 100 nM SB-269970 or 20 μM Roscovitine for 3 days, apoptosis assay was performed. Fluorescence from cleaved substrate of active caspase3/7 indicating apoptosis is shown. E. Quantification of apoptosis. Apoptotic cells showing caspase3/7 activity were counted in a confined area in 6 independent experiments and are represented as a fraction of total number of infected neurons. Data are shown as mean+SD. ** indicates statistical significance of p<0.01 (N=6; total analyzed cells 123 n 409, one-way ANOVA, followed by Bonferroni's post hoc test).

FIG. 7: Silencing of 5-HT7R reduces tau pathology in vivo.

A. Scheme showing experimental design: different AAVs (see FIG. 10A) were injected stereotactically into the pre-frontal cortex (PFC) of WT mice. After one month, various experiments were performed. B. Normalized mean slopes and representative examples of field excitatory postsynaptic potentials (fEPSPs) in brain slices from WT mice that were injected with AAV-eGFP, AAV-eGFP-Tau[R406W], AAV-eGFP-Tau[R406W]+AAV-scramble-shRNA, or AAV-eGFP-Tau[R406W]+AAV-5-HT7R-shRNA (see also FIG. 10B). The arrow denotes the time point at which five trains of TBS were applied. The insets represent averages of 30 fEPSPs recorded during 0-10 min before TBS (black, baseline) and 50-60 min after TBS (gray), respectively, in each group. C. Quantification of mean LTP levels recorded 50-60 min after TBS delivery in all groups. Data are presented as mean+SEM from the recorded slices: 8 slices from 6 mice for AAV-eGFP, 8 slices from 6 mice for AAV-eGFP-Tau[R406W], 7 slices from 5 mice for AAV-eGFP-Tau[R406W]+AAV-scramble-shRNA, and 8 slices from 6 mice for AAV-eGFP-Tau[R406W]+AAV-5-HT7R-shRNA. ** indicates statistical significance of p<0.01 (unpaired Student's t test). D. Staining of pre-frontal cortical slices of mice after AAV injection with phospho-specific tau antibody T205 (see also FIG. 10). E. Recency test with mice one month after injection of the indicated AAVs in the PFC. Quantification of discrimination and exploration time spent with less recent (L) and recent (R) object. Data are presented as mean+SEM (10≤N≤12).* indicates statistical significance of p<0.05, ** p<0.01 and ** p<0.001 (one-way ANOVA).

FIG. 8: Efficiency of G₁₂-shRNA.

A. Quantification of relative G₁₂ mRNA levels by quantitative Real-Time PCR in N1E-115 cells transfected with 3 different shRNAs against G₁₂. Cells transfected with src-shRNA were set to 100%. Data are represented as mean+SD (N=5). *** indicates statistical significance of p<0.001 (one-way ANOVA, followed by Dunnett's post hoc test). B. Western blot and quantification of G₁₂ protein levels in N1E-115 cells transfected scr-shRNA or G₁₂-shRNA #2. Signals were normalized to GAPDH expression and cells with scr-shRNA were set to 100%. Data are represented as mean+SD (N=6). *** indicates statistical significance of p<0.01 (Student's t-test).

FIG. 9: Tau tangles in N1E-115 cells.

Live cell imaging of N1E-115 cells transfected with eGFP-Tau[R406W]. Representative cells with (left panel) and without tau tangles (right panel) are shown.

FIG. 10: Validation of tau pathology in vivo.

A. Scheme of AAV constructs used for stereotactic injections in mice prefrontal cortex. B. Quantification of relative 5-HT7R mRNA levels by quantitative Real-Time PCR in primary cortical neurons infected with AAVs endcoding 2 different shRNAs against 5-HT7R. Cells infected with AAV-src-shRNA were set to 100%. Data are represented as mean+SD (N=3). *** indicates statistical significance of p<0.001 (one-way ANOVA, followed by Dunnett's post hoc test). C. Staining of pre-frontal slices after injection of AAV-eGFP-Tau[R406W] (eGFP, green) with phospho-specific tau antibody T205 (ptau, red). Right panel shows secondary antibody control. D and E. Open field test with mice one month after injection of the indicated AAVs. No significant differences (n.s.) in the distance travelled (D) and the time spent in the center (C) or the periphery (P) of the arena (E) were observed. Data are presented as mean+SEM (10≤N≤12, one-way ANOVA).

FIG. 11: 5-HT7R inverse agonists decrease 5-HT7R-induced Tau aggregation in HEK293 Tau-BiFCs.

A. Scheme showing experimental design. B. Quantification of tau-BiFC fluorescence intensity in pcDNA or (HA)5-HT7R-transfected HEK293 Tau-BiFC cells upon treatment with DMSO or 50 μM SB-269970, Amisulpride, Clozapine, Lurasidone, Miaserine or Tiapride for 24 h. Signals are normalized to DMSO-treated pcDNA-transfected cells. Data are represented as means+SDs (N=3). *** indicates statistical significance of p<0.001, n.s. not statistically different (one-way ANOVA, followed by Turkey's post hoc test). C. Representative maximum intensity projections of HEK293 Tau-BiFC cells transfected with control vector (mCerulean, blue) or 5-HT7R (CFP-5-HT7R, blue) upon the indicated treatment. Tangles of Venus-Tau (VN-Tau, yellow) presented in 5-HT7R-transfected cells disappeared after treatment with SB-269970 and amisulpride. D. Representative Western blot and quantification of HEK293 Tau-BiFC transfected with pcDNA or (HA)5-HT7R upon treatment with DMSO, SB-269970, Amisulpride, Clozapine, Lurasidone, Miaserine, Vortioxetine or Tiapride for 24 h. Membranes were probed with total Tau (Abcam) and GAPDH antibody (Millipore). Signals are normalized to DMSO-treated pcDNA-transfected cells. Data are represented as means+SDs (N=3). ** indicates statistical significance of p<0.01, *** p<0.001, n.s. not statistically different (one-way ANOVA, followed by Turkey's post hoc test). E. Dose-response curve for inhibition of Tau aggregation by Amisulpride. HEK293 Tau-BiFC cells were transfected with (HA)5-HT7R and treated with the indicated concentration of Amisulpride.

FIG. 12: 5-HT7R-induced Tau tangle formation and apoptosis in eGFP-Tau[R406W]-infected primary cortical neurons is reduced after treatment with Amisulpride.

A. Scheme showing experimental design. Primary cortical neurons were infected with AAV-eGFP-Tau[R406W] at DIV 10 and treated with H₂O, 100 nM SB-269970, 50 μM Amisulpride, 1 μM Vortioxetine or 50 μM Tiapride for three days. Neurons were analyzed at DIV 13. B. Representative images of neurons infected with AAV-eGFP-Tau[R406W] (green) and treated as indicated. Arrowheads indicate cells that were counted as tangle-positive. C. Quantification of Tau tangles. The number of tangle-positive neurons was counted in a confined area in at least four independent experiments and is represented as a fraction of the total number of infected neurons. Data are shown as mean+SEM. **** indicates statistical significance of p<0.0001, n.s. not statistically different (4≤N≤8; one-way ANOVA, Dunnett's post hoc test). D. Representative Western blot with neuronal lysates after sarkosyl fractionation. Tau signals in soluble, sarkosyl-soluble and sarkosyl-insoluble fractions (i.e. Tau-tangles) were detected by HRP-GFP antibody. GAPDH expression was detected in a soluble fraction. E. Quantification of apoptosis. Apoptotic cells showing caspase3/7 activity were counted in a confined area in at least four independent experiments and are represented as a fraction of total number of infected neurons. Data are shown as means+SEMs. **** indicates statistical significance of p<0.0001; n.s., not statistically different (4≤N≤8; one-way ANOVA, Dunnett's post hoc test).

FIG. 13: Amisulpride injections ameliorates Tau[R406W]-induced Tau hyperphosphorylation and memory impairments.

A. Scheme showing experimental design: AAV-eGFP-Tau[R406W] was injected stereotactically into the prefrontal cortex (PFC) of WT mice. After 3 weeks of recovery time, animals were intraperitoneally injected with either Vehicle or Amisulpride once daily for 16 days. B. Staining of prefrontal cortical slices of mice after AAV injection and treatment with phospho-specific Tau antibody T205. Representative overview image and magnification are shown. C. Quantification of the mean intensities of eGFP-Tau and pTau over the whole slice and calculation of the pTau/Tau ratio. Data are shown as mean+SEM (N=5). * indicates statistical significance of p<0.05 (one-tailed unpaired t-test). D. Results of test for temporal order recognition memory (recency test). Quantification of exploration time spent with less recent (L) and recent (R) object and discrimination index. Data are presented as mean+SEM (9≥N≥7). * indicates statistical significance of p<0.05, ** p<0.01; n.s., not statistically different (paired t-test and Wilcoxon signed rank test were applied to analyze exploration time, one-way ANOVA was applied to analyze the discrimination ratio).

### DETAILED DESCRIPTION OF THE INVENTION

Although the present invention is described in detail below, it is to be understood that this invention is not limited to the particular methodologies, protocols and reagents described herein as these may vary. It is also to be understood that the terminology used herein is for the purpose of describing particular embodiments only, and is not intended to limit the scope of the present invention which will be limited only by the appended claims. Unless defined otherwise, all technical and scientific terms used herein have the same meanings as commonly understood by one of ordinary skill in the art.

In the following, the elements of the present invention will be described. These elements are listed with specific embodiments, however, it should be understood that they may be combined in any manner and in any number to create additional embodiments. The variously described examples and preferred embodiments described throughout the specification should not be construed to limit the present invention to only the explicitly described embodiments. This description should be understood to support and encompass embodiments which combine the explicitly described embodiments with any number of the disclosed and/or preferred elements. Furthermore, any permutations and combinations of all elements described herein should be considered disclosed by the description of the present application unless the context indicates otherwise.

Throughout this specification and the claims which follow, unless the context requires otherwise, the word “comprise”, and variations such as “comprises” and “comprising”, will be understood to imply the inclusion of a stated member, integer or step or group of members, integers or steps but not the exclusion of any other member, integer or step or group of members, integers or steps although in some embodiments such other member, integer or step or group of members, integers or steps may be excluded, i.e. the subject-matter consists in the inclusion of a stated member, integer or step or group of members, integers or steps. When used herein the term “comprising” can be substituted with the term “containing” or “including” or sometimes when used herein with the term “having”. When used herein “consisting of” excludes any element, step, or ingredient not specified.

The terms “a” and “an” and “the” and similar reference used in the context of describing the invention (especially in the context of the claims) are to be construed to cover both the singular and the plural, unless otherwise indicated herein or clearly contradicted by context. Recitation of ranges of values herein is merely intended to serve as a shorthand method of referring individually to each separate value falling within the range. Unless otherwise indicated herein, each individual value is incorporated into the specification as if it were individually recited herein.

All methods described herein can be performed in any suitable order unless otherwise indicated herein or otherwise clearly contradicted by context. The use of any and all examples, or exemplary language (e.g., “such as”), provided herein is intended merely to better illustrate the invention and does not pose a limitation on the scope of the invention otherwise claimed. No language in the specification should be construed as indicating any non-claimed element essential to the practice of the invention.

Unless otherwise indicated, the term “at least” preceding a series of elements is to be understood to refer to every element in the series. The term “at least one” refers to one or more such as two, three, four, five, six, seven, eight, nine, ten and more. Those skilled in the art will recognize, or be able to ascertain using no more than routine experimentation, many equivalents to the specific embodiments of the invention described herein. Such equivalents are intended to be encompassed by the present invention.

The term “and/or” wherever used herein includes the meaning of “and”, “or” and “all or any other combination of the elements connected by said term”.

When used herein “consisting of” excludes any element, step, or ingredient not specified in the claim element. When used herein, “consisting essentially of” does not exclude materials or steps that do not materially affect the basic and novel characteristics of the claim.

The term “including” means “including but not limited to”. “Including” and “including but not limited to” are used interchangeably.

The term “about” means plus or minus 10%, preferably plus or minus 5%, more preferably plur or minus 2%, most preferably plus or minus 1%.

Throughout the description and claims of this specification, the singular encompasses the plural unless the context otherwise requires. In particular, where the indefinite article is used, the specification is to be understood as contemplating plurality as well as singularity, unless the context requires otherwise.

It should be understood that this invention is not limited to the particular methodology, protocols, material, reagents, and substances, etc., described herein and as such can vary. The terminology used herein is for the purpose of describing particular embodiments only, and is not intended to limit the scope of the present invention, which is defined solely by the claims.

Several documents are cited throughout the text of this specification. Each of the documents cited herein (including all patents, patent applications, scientific publications, manufacturers specifications, instructions, etc.), whether supra or infra, are hereby incorporated by reference in their entirety. Nothing herein is to be construed as an admission that the invention is not entitled to antedate such disclosure by virtue of prior invention. To the extent the material incorporated by reference contradicts or is inconsistent with this specification, the specification will supersede any such material.

The content of all documents and patent documents cited herein is incorporated by reference in their entirety.

A better understanding of the present invention and of its advantages will be gained from the examples, offered for illustrative purposes only. The examples are not intended to limit the scope of the present invention in any way.

### Compound

In general, serotonin receptors (5-HTRs) are divided into seven groups based on their structure, function, cellular response and distribution. They are mainly located in the hippocampus and prefrontal cortex of the brain (Strac et al., Transl Neurosci. 2016; 7(1): 35-492). In particular, the 5-HT7 receptor is coupled to two heterotrimeric G-proteins, GαS and Gα12 to facilitate formation of cyclic adenosine monophosphate (cAMP) by activating adenylyl cyclase and to activate small GTPases of the Rho family. In detail, when the 5-HT7 receptor is activated by serotonin, it sets off a cascade of events starting with release of the stimulatory G protein Gas from the GPCR complex. Gas in turn activates adenylate cyclase which increases intracellular levels of the second messenger cAMP.

In the present invention, it was found that the 5-HT7R activity induces tau-related pathomechanisms, including tau phosphorylation, tangle formation and neurotoxicity in a recombinant system as well as in primary cortical neurons and acute brain slices from mice.

Thus, by the present invention the importance of 5-HT7R signaling in tau-related neurodegeneration has been demonstrated. 5-HT7R-induced tau phosphorylation may be sufficient to promote the formation of sacrosyl-insoluble tau aggregates and obvious tangle-like structure within the cytoplasm. It was further shown that it may easily be envisioned that increased tau phosphorylation and tangle formation induced by 5-HT7R activity might reduce the capacity to degrade tau protein within cells. The increased number of tangle-like structures might additionally act as a “seed” for unphosphorylated, soluble tau species. Not only phosphorylated but also total tau levels may therefore increase.

Further, CDK5 as a critical mediator being responsible for 5-HT7R signaling upon tau has also been identified. Clear evidence has been found for an interaction of CDK5 with 5-HT7R through co-localization, FRET and co-precipitation. Genetic inhibition of CDK5, but not GSK3β activity, may abolish 5-HT7R elicited effect on tau phosphorylation in cells, demonstrating that Gα12 activation is not necessary for 5-HT7R-induced tau phosphorylation, thereby also speaking against a Gα12-mediated GSK3β activation. The same may apply to GαS, which may also not influence tau phosphorylation. An involvement of CDK5 activity in 5-HT7R signaling may clearly be emphasized.

Thus, a significant contribution of the 5-HT7R with regard to tau pathology has been demonstrated, thereby identifying a new putative molecular target from the family of serotonin receptors and, by now, the only one influencing tau deposition.

Most importantly, it has been found within the present invention that by blocking the basal activity of said 5-HT7R with an antagonist, hyperphosphorylation of the tau protein is prevented and through that mechanism the formation of tau-tangles in nerve cells is inhibited.

In particular, the 5-HT7 receptor antagonist may be used in preventing or treating a tauopathy by inhibiting both the receptor-induced phosphorylation and thus the accumulation of tau proteins.

Thereby, said 5-HT7 receptor antagonist of the present invention is able to halt the progression of a tauopathy or inhibit the development of a tauopathy at all and does not only treat the symptoms which are caused by said disease (e.g. cognitive functions, psychosis and behavioral problems).

In general, a tauopathy belongs to a class of neurodegenerative diseases associated with the pathological aggregation of tau protein in neurofibrillary or gliofibrillary tangles in the human brain. The tau protein undergoes several post-translational modifications, including acetylation, myristoylation with phosphorylation being the major modification of tau and its cellular functions. In tauopathies, tau aggregates are found to be extensively phosphorylated at several serine, threonine or tyrosine residues. Hyperphosphorylation of tau decreases its ability to bind to microtubules thereby disturbing transport of cargos and induces the accumulation of tau within the cell.

Thus, the term tauopathy encompasses both the loss-of-function effects on the microtubules and the gain-of-function effects of the toxic tau species. In detail, the consequences of tau hyperphosphorylation include not only loss of its function to bind and stabilize microtubule but also a gain of neurotoxic properties by tau aggregation into neurofibrillary tangles.

In this context and as used throughout the entire description, the term “prevent/preventing a tauopathy” refers to completely inhibiting the development of a tauopathy. By using a 5-HT7 receptor antagonist or a pharmaceutical composition comprising the 5-HT7 receptor antagonist, such antagonist or such pharmaceutical composition inhibits receptor-induced (hyper)-phosphorylation of a microtubule-associated protein known as tau protein, thereby preventing that the tau protein dissociates from microtubules and forms aggregates in an insoluble form, which are called tangles. Thus, the 5-HT7 receptor antagonist or the pharmaceutical composition comprising the 5-HT7 receptor antagonist inhibits the accumulation of phosphorylated tau proteins, thereby preventing that a tauopathy might occur anyway.

The term “treat/treating a tauopathy” as used throughout the entire description refers to halting the progression of a tauopathy in a subject which has been suffering from a tauopathy when the treatment with a 5-HT7 receptor antagonist or with a pharmaceutical composition comprising the 5-HT7 receptor antagonist has been initiated. Thereby, the 5-HT7 receptor antagonist or the pharmaceutical composition comprising the 5-HT7 receptor antagonist of the present invention prevents further receptor-induced (hyper)-phosphorylation of a tau protein, thereby preventing that the protein dissociates from microtubules and forms aggregates in an insoluble form, which are called tangles. Thus, the 5-HT7 receptor antagonist or the pharmaceutical composition comprising the 5-HT7 receptor antagonist reduces the accumulation of phosphorylated tau proteins, thereby halting the progression of a tauopathy.

There are a number of specific tauopathies, each of which vary by the distribution and morphological appearances of the protein-containing inclusions, as well as the relative burden of pathology affecting neurons and neuronal processes versus glial and glial processes. Tauopathies may present either with dementia, parkinsonism, or both.

In a preferred embodiment tauopathy is a dementia-associated tauopathy. Dementia-associated tauopathy refers to a tauopathy, whereby the patient suffering from said tauopathy also suffers from dementia. Dementia is a broad category of brain diseases that causes a long-term and often gradual decrease of the cognitive and memory functions that is great enough to affect a person's daily functioning.

Dementia-associated tauopathy may be selected from the group consisting of Alzheimer's disease, frontotemporal dementia, primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), Huntington disease, glial globular tauopathy. In a preferred embodiment dementia-associated tauopathy is Alzheimer's disease or frontotemporal dementia, more preferably Alzheimer's disease.

Alzheimer's disease accounts for 50% to 70% of all cases of dementia, being characterized by short-term memory loss and word-finding difficulties. In Alzheimer's disease, it is well established that filamentous tau protein deposits form within nerve cells that degenerate and that a good correlation exists between the number of tau deposits and the presence of dementia (Goedert et al, 1997, The Neuroscientist. 3, 131-141; Braak and Braak, 1991, Acta Neuropathologica. 82, 239-259; Arriagada et al, 1992, Neurology, 42(3) 631).

Therefore, in a preferred embodiment the present invention may comprise a 5-HT7R antagonist for use in treating or preventing a dementia-associated tauopathy, wherein said dementia-associated tauopathy is selected from the group consisting of Alzheimer's disease, frontotemporal dementia, primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), Huntington disease, glial globular tauopathy, preferably Alzheimer's disease or frontotemporal dementia, more preferably Alzheimer's disease.

In another preferred embodiment tauopathy may be selected from the group consisting of Alzheimer's disease, frontotemporal dementia, primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), Huntington disease, glial globular tauopathy, amyotrophic lateral sclerosis (ALS), Parkinson's disease, spinal muscular atrophy (SMA), cerebral amyloid angiopathy (CAA), preferably Alzheimer's disease or frontotemporal dementia, more preferably Alzheimer's disease.

Thus, the present invention may also comprise a 5-HT7R antagonist for use in treating or preventing a tauopathy, wherein said tauopathy is selected from the group consisting of Alzheimer's disease, frontotemporal dementia, primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), Huntington disease, glial globular tauopathy, amyotrophic lateral sclerosis (ALS), Parkinson's disease, spinal muscular atrophy (SMA), cerebral amyloid angiopathy (CAA), preferably Alzheimer's disease or frontotemporal dementia, more preferably Alzheimer's disease.

The term “antagonist” as used herein refers to two classes of antagonists. The first class refers to the neutral antagonists, which bind the receptor and have no intrinsic activity but will block the activity of agonists or inverse agonists. In this context, an agonist mimics the effects of the endogenous ligand, which is serotonin at the 5HT7-receptor, thus activating the receptor to produce biological response. Thus, in the context of the present invention at the 5-HT7 receptor, the cAMP level may increase.

The second class of antagonists refers to inverse agonists, which inhibit the constitutive (basal) activity of the receptor, producing functional effects opposite to those of agonists. Thus, in the context of the present invention at the 5-HT7 receptor, cAMP level may decrease. An inverse agonist may bind to the same receptor-binding site as an agonist.

In a preferred embodiment the 5-HT7 receptor antagonist is an antagonist with inverse agonist properties, thus acting as an inverse agonist. In another preferred embodiment, said inverse agonist targets and blocks the basal (constitutive) activity of 5-HT7R.

In 2004 after detailed pharmacological analysis, SB-269970 was declared as a 5-HT7R antagonist with full inverse agonist properties (Mahe et al. 2004, Eur J Pharmacol, 495(2-3):97-102). The same applies inter alia to Clozapine, for which also evidence for 5-HT7R inverse agonistic effects has been presented (Thomas et al., 1998, British Journal of Pharmacology 124, 1300-1306).

The term “alkyl” refers to a monoradical of a saturated straight or branched hydrocarbon. Preferably, the alkyl group comprises from 1 to 6 carbon atoms. For example the term “(C₁-C₆) alkyl” means that the respective alkyl group may comprise 1, 2, 3, 4, 5, or 6 carbon atoms. Exemplary alkyl groups include methyl, ethyl, propyl, iso-propyl, butyl (e.g. n-butyl, iso-butyl, tert-butyl), pentyl (e.g., n-pentyl, iso-pentyl, sec-pentyl, neo-pentyl), 1,2-dimethyl-propyl, iso-amyl, n-hexyl, iso-hexyl, sec-hexyl, 2,2-dimethylbutyl, n-heptyl, and the like. A “substituted alkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the alkyl group are replaced with a substituent other than hydrogen (when more than one hydrogen atom is replaced the substituents may be the same or different). Preferably, the substituent other than hydrogen is a 1^{st} level substituent, a 2^{nd} level substituent, or a 3^{rd} level substituent as specified herein, such as halogen or optionally substituted aryl. Examples of a substituted alkyl include trifluoromethyl, 2,2,2-trichloroethyl, arylalkyl (also called “aralkyl”, e.g., benzyl, chloro(phenyl)methyl, 4-methylphenylmethyl, (2,4-dimethylphenyl)methyl, o-fluorophenylmethyl, 2-phenylpropyl, 2-, 3-, or 4-carboxyphenylalkyl), or heteroarylalkyl (also called “heteroaralkyl”).

The term “cycloalkyl” refers to a monoradical of a saturated, non-aromatic cyclical hydrocarbon. In one embodiment, the cycloalkyl group has 1, 2, or more (preferably 1 or 2) double bonds. Preferably, the alkyl group comprises from 3 to 6 carbon atoms. “For example the term “(C₃-C₆)cycloalkyl” means that the respective alkyl group may comprise 3, 4, 5, or 6 carbon atoms. Exemplary cycloalkyl groups include cyclopropyl, cyclobutyl, cyclopentyl, cyclohexyl and the like. A “substituted cycloalkyl” means that one or more (such as 1 to the maximum number of hydrogen atoms bound to an alkyl group, e.g., 1, 2, 3, 4, 5, 6, 7, 8, 9, or up to 10, such as between 1 to 5, 1 to 4, or 1 to 3, or 1 or 2) hydrogen atoms of the cycloalkyl group are replaced with a substituent other than hydrogen (when more than one hydrogen atom is replaced the substituents may be the same or different). “—(C₃-C₈)cycloalkyl-(C₁-C₆)alkyl” means (C₃-C₈)cycloalkyl substituted with (C₁-C₆)alkyl.

The term “alkenyl” refers to a monoradical of an unsaturated straight or branched hydrocarbon having at least one carbon-carbon double bond. For example, the term “(C₂-C₆) alkenyl” means that the respective alkenyl group may comprise 1, 2, 3, 4, 5, or 6 carbon atoms. Generally, the maximum number of carbon-carbon double bonds in the alkenyl group can be equal to the integer which is calculated by dividing the number of carbon atoms in the alkenyl group by 2 and, if the number of carbon atoms in the alkenyl group is uneven, rounding the result of the division down to the next integer. For example, for an alkenyl group having 9 carbon atoms, the maximum number of carbon-carbon double bonds is 4. Preferably, the alkenyl group has 1 carbon-carbon double bond. Preferably, the alkenyl group comprises from 2 to 6 carbon atoms, such as from 2 to 4 or 2 carbon atoms, i.e., 2, 3, 4, 5, or 6 carbon atoms, more preferably 2 to 4 carbon atoms. Thus, in a preferred embodiment, the alkenyl group comprises from 2 to 6 carbon atoms and 1 or 2, carbon-carbon double bonds, more preferably it comprises 2 to 4 carbon atoms and 1 carbon-carbon double bond. The carbon-carbon double bond(s) may be in cis (Z) or trans (E) configuration. Exemplary alkenyl groups include ethenyl (i.e., vinyl), 1-propenyl, 2-propenyl (i.e., allyl), 1-butenyl, 2-butenyl, 3-butenyl, 1-pentenyl, 2-pentenyl, 3-pentenyl, 4-pentenyl, 1-hexenyl, 2-hexenyl, 3-hexenyl, 4-hexenyl, 5-hexenyl, 1, and the like. If an alkenyl group is attached to a nitrogen atom, the double bond cannot be alpha to the nitrogen atom.

The term “aryl” or “aromatic ring” refers to a monoradical of an aromatic cyclic hydrocarbon. Preferably, the aryl group contains 6 to 10 (e.g., 6 to 10, such as 6, or 10) carbon atoms which can be arranged in one ring (e.g., phenyl) or two or more condensed rings (e.g., naphthyl). For example the term “(C₆-C₁₀)aryl” means that the respective aryl group may comprise 6 to 10 carbon atoms. Exemplary aryl groups include, phenyl, indenyl, naphthyl, azulenyl, fluorenyl, anthryl, and phenanthryl. Preferably, “aryl” refers to a monocyclic ring containing 6 carbon atoms or an aromatic bicyclic ring system containing 10 carbon atoms. Preferred examples are phenyl and naphthyl. A “substituted aryl” means that one or more (such as 1 to the maximum number of hydrogen atoms bound to an aryl group, e.g., 1, 2, 3, 4, 5, 6, 7, 8, 9, or up to 10, such as between 1 to 5, 1 to 4, or 1 to 3, or 1 or 2) hydrogen atoms of the aryl group are replaced with a substituent other than hydrogen (when more than one hydrogen atom is replaced the substituents may be the same or different).

The term “heteroaryl” or “heteroaromatic ring” means an aryl group as defined above in which one or more carbon atoms in the aryl group are replaced by heteroatoms of 0, S, or N. Preferably, heteroaryl refers to a five or six-membered aromatic monocyclic ring wherein 1, 2, or 3 carbon atoms are replaced by the same or different heteroatoms of O, N, or S. For example the term “(C₅-C₁₀)heteroaryl” means that the heteroaryl group is based on an aryl group comprising 5 to 10 carbon atoms wherein one or more carbon atoms are replaced by heteroatoms of O, S, or N. Alternatively, it means an aromatic bicyclic or tricyclic ring system wherein 1, 2, 3, 4, or 5 carbon atoms are replaced with the same or different heteroatoms of O, N, or S. Preferably, in each ring of the heteroaryl group the maximum number of O atoms is 1, the maximum number of S atoms is 1, and the maximum total number of O and S atoms is 2. Exemplary heteroaryl groups include furanyl, thienyl, oxazolyl, isoxazolyl, oxadiazolyl, pyrrolyl, imidazolyl, pyrazolyl, triazolyl, tetrazolyl, thiazolyl, isothiazolyl, thiadiazolyl, pyridyl, pyrimidinyl, pyrazinyl, triazinyl, benzofuranyl, indolyl, isoindolyl, benzothienyl, 1H-indazolyl, benzimidazolyl, benzoxazolyl, indoxazinyl, benzisoxazolyl, benzothiazolyl, benzisothiazolyl, benzotriazolyl, quinolinyl, isoquinolinyl, benzodiazinyl, quinoxalinyl, quinazolinyl, benzotriazinyl, pyridazinyl, phenoxazinyl, thiazolopyridinyl, pyrrolothiazolyl, phenothiazinyl, isobenzofuranyl, chromenyl, xanthenyl, pyrrolizinyl, indolizinyl, indazolyl, purinyl, quinolizinyl, phthalazinyl, naphthyridinyl, cinnolinyl, pteridinyl, carbazolyl, phenanthridinyl, acridinyl, perimidinyl, phenanthrolinyl, and phenazinyl. Exemplary 5- or 6-membered heteroaryl groups include furanyl, thienyl, oxazolyl, isoxazolyl, oxadiazolyl, pyrrolyl, imidazolyl (e.g., 2-imidazolyl), pyrazolyl, triazolyl, tetrazolyl, thiazolyl, isothiazolyl, thiadiazolyl, pyridyl (e.g., 4-pyridyl), pyrimidinyl, pyrazinyl, triazinyl, and pyridazinyl. A “substituted heteroaryl” means that one or more (such as 1 to the maximum number of hydrogen atoms bound to a heteroaryl group, e.g., 1, 2, 3, 4, 5, 6, 7, 8, 9, or up to 10, such as between 1 to 5, 1 to 4, or 1 to 3, or 1 or 2) hydrogen atoms of the heteroaryl group are replaced with a substituent other than hydrogen (when more than one hydrogen atom is replaced the substituents may be the same or different).

The term “halogen” or “halo” means fluoro, chloro, bromo, or iodo. The term “hydroxy” means OH. The term “cyano” means the group —CN. The term “isocyano” means the group —NC.

As used herein and throughout the entire description, the term “optional” or “optionally” as used herein means that the subsequently described event, circumstance or condition may or may not occur, and that the description includes instances where said event, circumstance, or condition occurs and instances in which it does not occur.

Preferably, the 5-HT7 receptor antagonist has structure according to at least one of the general formula (I) to (VI), or is a pharmaceutically acceptable salt, prodrug, enantiomer, diastereomer, racemic mixture, crystalline form, amorphous, unsolved form or solvate of the general formula (I), (II), (Ill), (IV), (V) or (VI) as follows.

Formula (I):

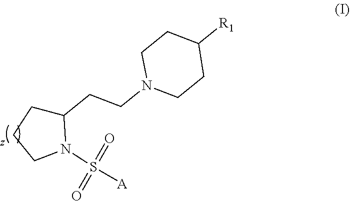

wherein in formula (I)
R₁ is —(C₁-C₆)alkyl, preferably methyl, (C₆-C₁₀)aryl, —O(C₆-C₁₀)aryl, or (C₅-C₁₀)heteroaryl,
which are optionally substituted with one or more substituents independently selected from the group consisting of: halogen, preferably —Cl, or (C₁-C₆)Alkyl;
A is (C₆-C₁₀)aryl, or (C₅-C₁₀)heteroaryl,
which are optionally substituted with one or more substituents independently selected from the group consisting of: halogen, —OH, (C₁-C₆)alkyl, or —O(C₁-C₆)alkyl, preferably the at least one substituent is a meta-substituent;
z is 1.

Formula (II):

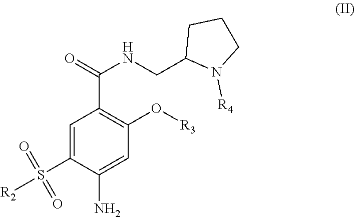

wherein in formula (II)
R₂ and R₃ are independently selected from hydrogen, (C₁-C₆)alkyl, preferably methyl or ethyl, most preferably ethyl;
R₄ is hydrogen, (C₁-C₆)alkyl, preferably methyl or ethyl, most preferably ethyl, or (C₂-C₆)alkenyl.

Formula (III):

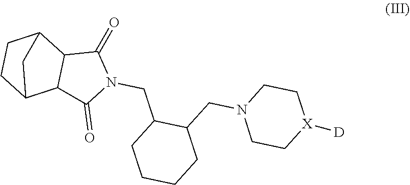

wherein in formula (III)

### X is N or CH;

D is hydrogen, (C₅-C₁₀)heteroaryl, preferably selected from

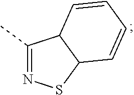

(C₆-C₁₀)aryl, —O(C₆-C₁₀)aryl, or —S(C₆-C₁₀)aryl,
which are optionally substituted with one or more substituents independently selected from the group consisting of: halogen, (C₁-C₆)alkyl, or —OH.

Formula (IV):

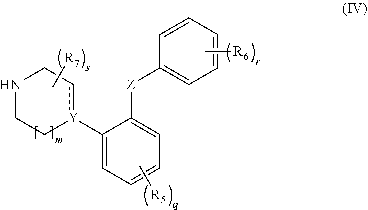

wherein in formula (IV)

### Z is O or S;

### Y is N, C or CH;

s is an integer in the range 0 to 8;
q is an integer in the range 0 to 4;
r is an integer in the range 0 to 5;
m is 1 or 2;
The dotted line represents an optional bond; if the optional bond, indicated as dotted line, is not present, the carbon atom, attached to the optional bond, neighboring Y is saturated with hydrogen and Y is CH or N. If the optional bond is present, Y may be N or C.
R₇ is hydrogen, —(C₁-C₆)alkyl, or two R7 attached to the same carbon atom may form a 3 to 6 membered spiro attached cyclo-alkyl;
R₅ is hydrogen, halogen, cyano, —NO₂, (C₁-C₆)alkenyl, (C₁-C₆)alkyl, —O(C₁-C₆)alkyl, —OH, hydroxy(C₁-C₆)alkyl, halo(C₁-C₆)alkyl, (C₃-C₈)cycloalkyl, (C₃-C₈)cycloalkyl-(C₁-C₆)alkyl, acyl, —CO₂(C₁-C₆)alkyl, —(C₆-C₁₀)aryl, —SO₂(C₁-C₆)alkyl, or —NRₓR_{y};
R₆ is hydrogen, halogen, —CN, —NO₂, (C₁-C₆)alkenyl, (C₁-C₆)alkyl, —O(C₁-C₆)alkyl, —OH, hydroxy(C₁-C₆)alkyl, halo(C₁-C₆)alkyl, (C₃-C₈)cycloalkyl, C₃-C₈)cycloalkyl-(C₁-C₆)alkyl, acyl, —CO₂(C₁-C₆)alkyl, (C₆-C₁₀)aryl, —SO₂(C₁-C₆)alkyl, —NRₓR_{y}; —NRₓCO(C₁-C₆)alkyl, or —CONRₓR_{y};

wherein each Rₓ and R_{y} is independently selected from hydrogen, (C₁-C₆)alkyl, (C₃-C₈)cycloalkyl, (C₃-C₈)cycloalkyl-(C₁-C₆)alkyl, or (C₆-C₁₀)aryl;

or Rₓ and R_{y}, together with the nitrogen to which they are attached from a 3 to 7 membered ring which optionally contains one further heteroatom.

Formula (V):

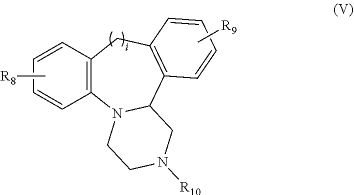

wherein in formula (V)
R₈ and R₉ are independently selected from hydrogen, halogen, —OH, —OCO(C₁-C₆)alkyl, (C₁-C₆)alkyl, —O(C₁-C₆)alkyl, or —CF₃;
R₁₀ is hydrogen, (C₁-C₆)alkyl, or —(C₁-C₆)alkylaryl;
i is 0 or 1;

Formula (VI):

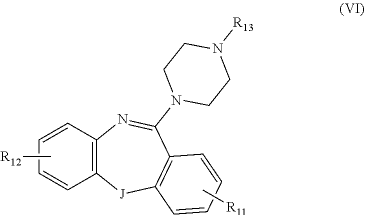

wherein in formula (VI)
R₁₁ and R₁₂ are independently selected from hydrogen, halogen, —OH, (C₁-C₆)alkyl, —CF₃, —O(C₁-C₆)alkyl, —NO₂, —NRₒR_{z}, —SO₂NRₒR_{z}, or —S(C₁-C₆)alkyl;
wherein each Rₒ and R_{z} is independently selected from hydrogen, (C₁-C₆)alkyl;
R₁₃ is hydrogen, allyl, (C₁-C₆)alkyl, preferably (C₁-C₃)alkyl, —O(C₁-C₆)alkyl, or hydroxy(C₁-C₆)alkyl, preferably hydroxy(C₁-C₃)alkyl;

### J is S or NH.

More preferably, the 5-HT7 receptor antagonist of the present invention may be selected from the group consisting of SB-269970, Amisulpride, Lurasidone, Vortioxetine, Mianserin, Clozapine.

Most preferably, the 5-HT7 receptor antagonist is SB-269970.

The in vitro experiments of the present invention demonstrate that blocking the basal activity of the 5-HT7R using an antagonist, such as SB-269970, leads to a reduced accumulation of p-tau. In detail, a neuroblastoma cell line transfected with a plasmid expressing mutated tau and 5-HT7R, shows increased protein levels of tau, p-tau and tau aggregates, whereas the simultaneous addition of SB-269970 to the medium prevented this effect. This demonstrates that a treatment with SB-269970 prevents both tangle formation as well as tau hyperphosphorylation.

In a preferred embodiment of the present invention the 5-HT7 receptor antagonist may be used in preventing or treating a tauopathy, wherein the 5-HT7 receptor antagonist is SB-269970.

5-HT7 receptor antagonists having a structure according to at least one of the general formula (I) to (VI), or is a pharmaceutically acceptable salt, prodrug, enantiomer, diastereomer, racemic mixture, crystalline form, amorphous, unsolved form or solvate of the general formula (I), (II), (Ill), (IV), (V) or (VI), in particular such as Amisulpride, Lurasidone, Vortioxetine, Mianserin and Clozapine are compounds being approved antidepressants and antipsychotics in the prior art, that act as antagonists of the 5-HT7R such as SB-269970. Until now they have been applied in several other diseases such as schizophrenia, bipolar disorder, depressive disorder or depression in general. However, it has now been surprisingly found that 5-HT7R antagonists may be applied in preventing or treating a tauopathy.

In one embodiment said antagonist of the present invention may be a small organic molecule comprising at least two carbon atoms having a molecular weight in the range between 100 and 1000 Dalton (g/mol). The present invention may provide a 5-HT7 receptor antagonist comprising at least two, three, four, five, six, seven, eight, nine, ten, fifteen, twenty, twenty-five, thirty, thirty-five, forty, forty-five, fifty or more carbon atoms. The 5-HT7 receptor antagonist of the present invention may have a molecular weight in the range between 100 and 1000 Dalton, 150 to 800 Dalton, 170 to 600 Dalton, or 200 to 550 Dalton. Preferably, the 5-HT7R antagonist may comprise at least ten carbon atoms having a molecular weight in the range between 200 to 550 Dalton.

### Pharmaceutical Compositions

In a further aspect, the present invention provides a pharmaceutical composition comprising the 5-HT7 receptor antagonist as specified above. Further, said pharmaceutical composition may comprise the 5-HT7 receptor antagonist and one or more pharmaceutically acceptable excipients.

The compounds described in the present invention (in particular those specified above such as those of formula I, II, III, IV, V, and VI or a pharmaceutically acceptable salt, prodrug, enantiomer, diastereomer, racemic mixture, crystalline form, amorphous, unsolved form or solvate of the general formula I, II, III, IV, V or VI) are preferably administered to a patient in need thereof via a pharmaceutical composition. In one embodiment, the pharmaceutical composition comprises a compound as described above (e.g. having the general formula I, II, III, IV, V and VI, or a pharmaceutically acceptable salt, prodrug, enantiomer, diastereomer, racemic mixture, crystalline form, amorphous, unsolved form or solvate of the general formula I, II, III, IV, V or VI) and one or more pharmaceutically acceptable excipients.

The pharmaceutical composition may be administered to an individual by any route, such as enterally, parenterally or by inhalation.

The expressions “enteral administration” and “administered enterally” as used herein mean that the drug administered is taken up by the stomach and/or the intestine. Examples of enteral administration include oral and rectal administration. The expressions “parenteral administration” and “administered parenterally” as used herein mean modes of administration other than enteral administration, usually by injection or topical application, and include, without limitation, intravenous, intramuscular, intraarterial, intrathecal, intracapsular, intraosseous, intraorbital, intracardiac, intradermal, intraperitoneal, transtracheal, subcutaneous, subcuticular, intraarticular, subcapsular, intracerebral, intracerebroventricular, subarachnoid, intraspinal, epidural and intrasternal administration (such as by injection and/or infusion) as well as topical administration (e.g., epicutaneous, or through mucous membranes (such as buccal, sublingual or vaginal)). However, preferred is an enterally or parenterally administration.

The compounds used in the present invention are generally applied in “pharmaceutically acceptable amounts” and in “pharmaceutically acceptable preparations”. Such compositions may contain salts, buffers, preserving agents, carriers and optionally other therapeutic agents.

The term “excipient” when used herein is intended to indicate all substances in a pharmaceutical composition which are not active ingredients (e.g., which are therapeutically inactive ingredients that do not exhibit any therapeutic effect in the amount/concentration used), such as, e.g., carriers, binders, lubricants, thickeners, surface active agents, preservatives, emulsifiers, buffers, flavoring agents, colorants, or antioxidants.

The pharmaceutical compositions comprising the 5-HT7 receptor antagonist described in the present invention may also comprise a pharmaceutically acceptable carrier. As used herein, “pharmaceutically acceptable carrier” includes any and all solvents, dispersion media, coatings, isotonic and absorption delaying agents, and the like that are physiologically compatible. The “pharmaceutically acceptable carrier” may be in the form of a solid, semisolid, liquid, or combinations thereof. Preferably, the carrier is suitable for enteral (such as oral) or parenteral administration (such as intravenous, intramuscular, subcutaneous, spinal or epidermal administration (e.g., by injection or infusion)). Depending on the route of administration, the active compound, i.e., the 5-HT7 receptor antagonist used in the present invention, either alone or in combination with one or more additional active compounds, may be coated in a material to protect the active compound(s) from the action of acids and other natural conditions that may inactivate the active compound.

Thus, a pharmaceutical composition of the present invention may comprise the 5-HT7 receptor antagonist, one or more pharmaceutically acceptable excipient(s) and a pharmaceutically acceptable carrier. Additionally, a pharmaceutical composition of the present invention may comprise the 5-HT7 receptor antagonist, one or more pharmaceutically acceptable excipient(s), a pharmaceutically acceptable carrier and at least one additional active compound, thus being used as a combined preparation.

Examples of suitable aqueous and non-aqueous carriers which may be employed in the pharmaceutical compositions used according to the present invention include water (e.g., water for injection), ethanol, polyols (such as glycerol, propylene glycol, polyethylene glycol, and the like), aqueous solutions of a salt, carbohydrate, sugar alcohol, or an amino acid (such as saline or an aqueous amino acid solution), and suitable mixtures and/or buffered forms thereof, injectable organic esters (such as ethyl oleate) and preferably oils, such as vegetable oils or olive oil). In case of inhalation a suitable carrier for the pharmaceutical composition may be e.g. captisol, sugar alcohol or any other aqueous component.

Pharmaceutically acceptable carriers include sterile aqueous solutions or dispersions and sterile powders for the extemporaneous preparation of sterile injectable solutions or dispersions. The use of such media and agents for pharmaceutically active compounds is known in the art. Except insofar as any conventional media or agent is incompatible with the active compound, use thereof in the pharmaceutical compositions used according to the present invention is contemplated.

In some embodiments, an additional active compounds may also be administered together with, before or after the compound used in the present invention (in particular those specified above such as those of formula I, II, III, IV, V, and VI or a pharmaceutically acceptable salt, prodrug, enantiomer, diastereomer, racemic mixture, crystalline form, amorphous, unsolved form or solvate of the general formula I, II, III, IV, V or VI). In one embodiment, the pharmaceutical composition described herein comprises a compound as described above (in particular those specified above such as those of formula I, II, III, IV, V, and VI or a pharmaceutically acceptable salt, prodrug, enantiomer, diastereomer, racemic mixture, crystalline form, amorphous, unsolved form or solvate of the general formula I, II, III, IV, V or VI) and at least one additional active compound. Also comprised herein is the pharmaceutical composition described herein comprises a compound as described above (in particular those specified above such as those of formula I, II, III, IV, V, and VI or a pharmaceutically acceptable salt, prodrug, enantiomer, diastereomer, racemic mixture, crystalline form, amorphous, unsolved form or solvate of the general formula I, II, III, IV, V or VI) and at least one additional active compound and one or more pharmaceutically acceptable excipients. Also comprised herein is the pharmaceutical composition described herein comprises a compound as described above (in particular those specified above such as those of formula I, II, III, IV, V, and VI or a pharmaceutically acceptable salt, prodrug, enantiomer, diastereomer, racemic mixture, crystalline form, amorphous, unsolved form or solvate of the general formula I, II, III, IV, V or VI) and at least one additional active compound and a pharmaceutically acceptable carrier.

The “additional active compound” (which is not a compound having formula I, II, III, IV, V, and VI or a pharmaceutically acceptable salt, prodrug, enantiomer, diastereomer, racemic mixture, crystalline form, amorphous, unsolved form or solvate of the general formula I, II, III, IV, V or VI) may be selected from any compound which may be used in the prevention or treatment of tauopathy. The additional active compound may induce an additive or synergistic therapeutic effect. The additional active compound used to prevent or treat a tauopathy may be a CDK5-inhibitor, preferably Roscovitine. Thus, the present invention comprises a pharmaceutical composition for use in preventing or treating a tauopathy comprising the 5-HT7 receptor antagonist as mentioned elsewhere herein and a CDK5-inhibitor, preferably Roscovitine.

The pharmaceutical composition may also comprise adjuvants such as preservatives, wetting agents, emulsifying agents, pH buffering agents, and dispersing agents. Prevention of the presence of microorganisms may be ensured by sterilization procedures and/or by the inclusion of various antibacterial and antifungal agents, for example, paraben, chlorobutanol, phenol sorbic acid, and the like. It may also be desirable to include isotonic agents, such as sugars, sodium chloride, and the like into the compositions. In addition, prolonged absorption of the injectable pharmaceutical form may be brought about by the inclusion of agents which delay absorption such as aluminum monostearate and gelatin.

Regardless of the route of administration selected, the active compounds (the 5-HT7 receptor antagonist), which may be used in a suitable hydrated form, and/or the pharmaceutical compositions used according to the present invention, are formulated into pharmaceutically acceptable dosage forms by conventional methods known to those of skill in the art (cf., e.g., Remington, “The Science and Practice of Pharmacy” edited by Allen, Loyd V., Jr., 22^{nd} edition, Pharmaceutical Sciences, September 2012; Ansel et al., “Pharmaceutical Dosage Forms and Drug Delivery Systems”, 7th edition, Lippincott Williams & Wilkins Publishers, 1999).

A pharmaceutical composition can be administered by a variety of methods known in the art. As will be appreciated by the skilled artisan, the route and/or mode of administration will vary depending upon the desired results. The pharmaceutical compositions containing one or more active compounds can be prepared with carriers that will protect the one or more active compounds against rapid release, such as a controlled release formulation, including implants, transdermal patches, and microencapsulated delivery systems. Biodegradable, biocompatible polymers can be used, such as ethylene vinyl acetate, polyanhydrides, polyglycolic acid, collagen, polyorthoesters, and polylactic acid. Methods for the preparation of such compositions are generally known to those skilled in the art. See, e.g., Sustained and Controlled Release Drug Delivery Systems, J. R. Robinson, ed., Marcel Dekker, Inc., New York, 1978.

To administer a compound used in the present invention by certain routes of administration, it may be necessary to coat the compound with, or co-administer the compound with, a material to prevent its inactivation. For example, the compound may be administered to an individual in an appropriate carrier, for example, liposomes, oil or a diluent. Pharmaceutically acceptable diluents include saline and aqueous buffer solutions. Liposomes include water-in-oil-in-water CGF emulsions as well as conventional liposomes (Strejan et al., J. Neuroimmunol. 7: 27(1984)).

Pharmaceutical compositions typically are sterile and stable under the conditions of manufacture and storage.

Dosage regimens are adjusted to provide the optimum desired response (e.g., a therapeutic response). For example, a single bolus may be administered, several divided doses may be administered over time or the dose may be proportionally reduced or increased as indicated by the exigencies of the therapeutic situation. It is especially advantageous to formulate parenteral compositions in dosage unit form for ease of administration and uniformity of dosage. Dosage unit form as used herein refers to physically discrete units suited as unitary dosages for the individuals to be treated; each unit contains a predetermined quantity of active compound calculated to produce the desired therapeutic effect in association with the required pharmaceutical carrier. The specification for the dosage unit forms used according to the present invention are dictated by and directly dependent on (a) the unique characteristics of the active compound and the particular therapeutic effect to be achieved, and (b) the limitations inherent in the art of compounding such an active compound for the treatment of sensitivity in individuals.

Generally, out of 100% (for the pharmaceutical formulations/compositions), the amount of active ingredient (in particular, the amount of the compound used according to the present invention, optionally together with other therapeutically active agents, if present in the pharmaceutical formulations/compositions) will range from about 0.01% to about 99%, preferably from about 0.1% to about 70% The amount of active ingredient, e.g., a compound used according to the present invention, in a unit dosage form and/or when administered to an individual or used in therapy, may range from about 0.1 mg to about 10000 mg (for example, from about 1 mg to about 5000 mg, such as from about 10 mg to about 2000 mg) per unit, administration or therapy.

Actual dosage levels of the active ingredients in the pharmaceutical compositions used according to the present invention may be varied so as to obtain an amount of the active ingredient which is effective to achieve the desired therapeutic response for a particular patient, composition, and mode of administration, without being toxic to the patient. The selected dosage level will depend upon a variety of pharmacokinetic factors including the activity of the particular compositions employed, the route of administration, the time of administration, the rate of excretion of the particular compound being employed, the duration of the treatment, other drugs, compounds and/or materials used in combination with the particular compositions employed, the age, sex, weight, condition, general health and prior medical history of the patient being treated, and like factors well known in the medical arts.

A physician or veterinarian having ordinary skill in the art can readily determine and prescribe the effective amount of the pharmaceutical composition required. For example, the physician or veterinarian could start with doses of the compounds used according to the present invention at levels lower than that required in order to achieve the desired therapeutic effect and gradually increase the dosage until the desired effect is achieved. In general, a suitable daily dose of a composition used according to the present invention will be that amount of the compound which is the lowest dose effective to produce a therapeutic effect. Such an effective dose will generally depend upon the factors described above. It is preferred that administration be oral, intravenous, intramuscular, intraperitoneal, or subcutaneous. It is even more preferred that administration be oral, intramuscular or intravenous. If desired, the effective daily dose of a pharmaceutical composition may be administered as two, three, four, five, six or more sub-doses administered separately at appropriate intervals throughout the day, optionally, in unit dosage forms. While it is possible for a compound used according to the present invention to be administered alone, it is preferable to administer the compound as a pharmaceutical formulation/composition.

For oral administration, the pharmaceutical composition used according to the present invention can take the form of, for example, tablets or capsules prepared by conventional means with pharmaceutical acceptable excipients such as binding agents (e.g., pregelatinised maize starch, polyvinylpyrrolidone, hydroxypropyl methylcellulose), fillers (e.g., lactose, microcrystalline cellulose, calcium hydrogen phosphate), lubricants (e.g., magnesium stearate, talc, silica), disintegrants (e.g., potato starch, sodium starch glycolate), or wetting agents (e.g., sodium lauryl sulphate). Liquid preparations for oral administration can be in the form of, for example, solutions, syrups, or suspensions, or can be presented as a dry product for reconstitution with water or other suitable vehicle before use. Such liquid preparation can be prepared by conventional means with pharmaceutically acceptable additives such as suspending agents (e.g., sorbitol, syrup, cellulose derivatives, hydrogenated edible fats), emulsifying agents (e.g., lecithin, acacia), non-aqueous vehicles (e.g., almond oil, oily esters, ethyl alcohol, fractionated vegetable oils), preservatives (e.g., methyl or propyl-p-hydroxycarbonates, sorbic acids). The preparations can also contain buffer salts, flavouring, coloring and sweetening agents as deemed appropriate.

In one embodiment, the compound is orally administered in a concentration of at most 1000 mg/kg body weight (such as at most 500 mg/kg body weight, at most 400 mg/kg body weight, at most 300 mg/kg body weight, at most 200 mg/kg body weight, at most 100 mg/kg body weight, at most 50 mg/kg body weight, at most 40 mg/kg body weight, at most 30 mg/kg body weight, at most 20 mg/kg body weight, at most 10 mg/kg body weight or less).

Oral formulation can include standard carriers such as pharmaceutical grades of mannitol, lactose, starch, magnesium stearate, sodium saccharine, cellulose, magnesium carbonate, etc.

The pharmaceutical composition used according to the present invention can be formulated for parenteral administration by injection, for example, by bolus injection, continuous infusion or intraperitoneal injection. In one embodiment, the compounds or compositions used according to the present invention may be administered by slow continuous infusion over a long period, such as more than 24 hours, in order to reduce toxic side effects. The administration may also be performed by continuous infusion over a period of from 2 to 24 hours, such as of from 2 to 12 hours. Such regimen may be repeated one or more times as necessary, for example, after 6 months or 12 months.

In one embodiment, the compound is parenterally administered (e.g., intravenously, intramuscularly, subcutaneously or by inhalation), in a concentration of at most 100 mg/kg body weight (such as at most 50 mg/kg body weight, at most 40 mg/kg body weight, at most 30 mg/kg body weight, at most 20 mg/kg body weight, at most 10 mg/kg body weight, at most 5 mg/kg body weight, at most 4 mg/kg body weight, at most 3 mg/kg body weight, at most 2 mg/kg body weight, at most 1 mg/kg body weight, at most 0.1 mg/kg body weight, at most 0.01 mg/kg body weight).

Formulations for injection can be presented in units dosage form (e.g., in phial, in multi-dose container), and with an added preservative. The pharmaceutical composition used according to the present invention can take such forms as suspensions, solutions or emulsions in oily or aqueous vehicles, and can contain formulatory agents such as suspending, stabilizing, or dispersing agents. Alternatively, the agent can be in powder form for constitution with a suitable vehicle (e.g., sterile pyrogen-free water) before use. Typically, compositions for intravenous administration are solutions in sterile isotonic aqueous buffer. Where necessary, the composition can also include a solubilizing agent and a local anesthetic such as lignocaine to ease pain at the site of the injection. Generally, the ingredients are supplied either separately or mixed together in unit dosage form, for example, as a dry lyophilized powder or water free concentrate in a hermetically sealed container such as an ampoule or sachette indicating the quantity of active agent. Where the composition is to be administered by infusion, it can be dispensed with an infusion bottle containing sterile pharmaceutical grade water or saline. Where the composition is administered by injection, an ampoule of sterile water for injection or saline can be provided so that the ingredients can be mixed prior to administration.

Dosage forms for the topical, transdermal or inhalation administration of compositions used according to the present invention may include powders, sprays, ointments, pastes, creams, lotions, gels, solutions, patches and inhalants. The active compound may be mixed under sterile conditions with a pharmaceutically acceptable carrier, and with any preservatives, buffers, or propellants which may be required.

The pharmaceutical composition used according to the invention can also, if desired, be presented in a pack, or dispenser device which can contain one or more unit dosage forms containing the active compound. The pack can for example comprise metal or plastic foil, such as blister pack. The pack or dispenser device can be accompanied with instruction for administration.

In summary, the pharmaceutical composition comprising the 5-HT7 receptor antagonist of the present invention may be used in preventing or treating a tauopathy by inhibiting both the receptor-induced phosphorylation and thus the accumulation of tau proteins. Thereby, said pharmaceutical composition comprising the 5-HT7 receptor antagonist of the present invention is able to halt the progression of a tauopathy or completely inhibit the development of a tauopathy and does not only treat the symptoms which are caused by said disease (e.g. cognitive functions, psychosis and behavioral problems).

In some embodiments, the present invention may comprise the pharmaceutical composition comprising the 5-HT7 receptor antagonist for use in treating or preventing a dementia-associated tauopathy, wherein said dementia-associated tauopathy is selected from the group consisting of Alzheimer's disease, frontotemporal dementia, primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), Huntington disease, glial globular tauopathy, preferably Alzheimer's disease or frontotemporal dementia, more preferably Alzheimer's disease.

In another embodiment, the present invention may also comprise the pharmaceutical composition comprising the 5-HT7 receptor antagonist for use in treating or preventing a tauopathy, wherein said tauopathy is selected from the group consisting of Alzheimer's disease, frontotemporal dementia, primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), Huntington disease, glial globular tauopathy, amyotrophic lateral sclerosis (ALS), Parkinson's disease, spinal muscular atrophy (SMA), cerebral amyloid angiopathy (CAA), preferably Alzheimer's disease or frontotemporal dementia, more preferably Alzheimer's disease.

1. A method for treating or preventing a tauopathy in a subject in need thereof, the method comprising administering to the subject a 5-HT7 receptor antagonist.

2. The method of claim 1, wherein the 5-HT7 receptor antagonist has a structure according to the following formula (I), 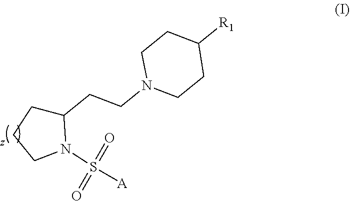
wherein in formula (I)
R₁ is —(C₁-C₆)alkyl, preferably methyl, (C₆-C₁₀)aryl, —O(C₆-C₁₀)aryl, or (C₅-C₁₀)heteroaryl,
which are optionally substituted with one or more substituents independently selected from the group consisting of: halogen, preferably —C₁, or (C₁-C₆)Alkyl;
A is (C₆-C₁₀)aryl, or (C₅-C₁₀)heteroaryl,
which are optionally substituted with one or more substituents independently selected from the group consisting of: halogen, —OH, (C₁-C₆)alkyl, or —O(C₁-C₆)alkyl, preferably the at least one substituent is a meta-substituent;
z is 1 or;
a structure according to formula (II)
wherein in formula (II) 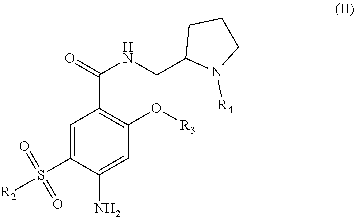
R₂ and R₃ are independently selected from hydrogen, (C₁-C₆)alkyl, preferably methyl or ethyl, most preferably ethyl;
R₄ is hydrogen, (C₁-C₆)alkyl, preferably methyl or ethyl, most preferably ethyl, or (C₂-C₆)alkenyl;
or a structure according to formula (III) 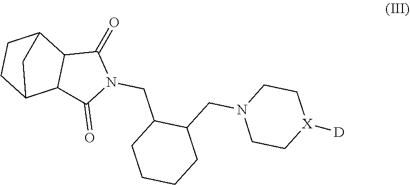
wherein in formula (III)
X is N or CH;
D is hydrogen, (C₅-C₁₀)heteroaryl, preferably selected from 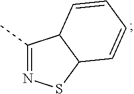
(C₆-C₁₀)aryl, —O(C₆-C₁₀)aryl, or —S(C₆-C₁₀)aryl,
which are optionally substituted with one or more substituents independently selected from the group consisting of: halogen, (C₁-C₆)alkyl, or —OH;
or a structure according to formula (IV) 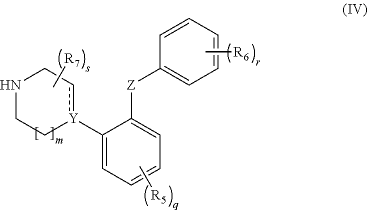
wherein in formula (IV)
Z is O or S;
Y is N, C or CH;
s is an integer in the range 0 to 8;
q is an integer in the range 0 to 4;
r is an integer in the range 0 to 5;
m is 1 or 2;
The dotted line represents an optional bond;
R₇ is hydrogen, —(C₁-C₆)alkyl, or two R₇ attached to the same carbon atom may form a 3 to 6 membered spiro attached cyclo-alkyl;
R₅ is hydrogen, halogen, cyano, —NO₂, (C₁-C₆)alkenyl, (C₁-C₆)alkyl, —O(C₁-C₆)alkyl, —OH, hydroxy(C₁-C₆)alkyl, halo(C₁-C₆)alkyl, (C₃-C₅)cycloalkyl, —(C₃-C₅)cycloalkyl-(C₁-C₆)alkyl, acyl, —CO₂(C₁-C₆)alkyl, —(C₆-C₁₀)aryl, —SO₂(C₁-C₆)alkyl, or —NRₓR_{y};
R₆ is hydrogen, halogen, —CN, —NO₂, (C₁-C₆)alkenyl, (C₁-C₆)alkyl, —O(C₁-C₆)alkyl, —OH, hydroxy(C₁-C₆)alkyl, halo(C₁-C₆)alkyl, (C₃-C₅)cycloalkyl, —(C₃-C₅)cycloalkyl-(C₁-C₆)alkyl, acyl, —CO₂(C₁-C₆)alkyl, (C₆-C₁₀)aryl, —SO₂(C₁-C₆)alkyl, —NRₓR_{y}; —NRₓCO(C₁-C₆)alkyl, or —CONRₓR_{y};
wherein each Rₓ and R_{y} is independently selected from hydrogen, (C₁-C₆)alkyl, (C₃-C₅)cycloalkyl, —(C₃-C₅)cycloalkyl-(C₁-C₆)alkyl, or (C₆-C₁₀)aryl;
or Rₓ and R_{y}, together with the nitrogen to which they are attached from a 3 to 7 membered ring which optionally contains one further heteroatom;
or a structure according to formula (V) 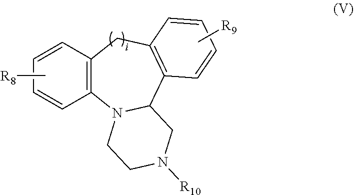
wherein in formula (V)
R₅ and R₉ are independently selected from hydrogen, halogen, —OH, —OCO(C₁-C₆)alkyl, (C₁-C₆)alkyl, —O(C₁-C₆)alkyl, or —CF₃;
R₁₀ is hydrogen, (C₁-C₆)alkyl, or —(C₁-C₆)alkylaryl;
i is 0 or 1;
or a structure according to formula (VI) 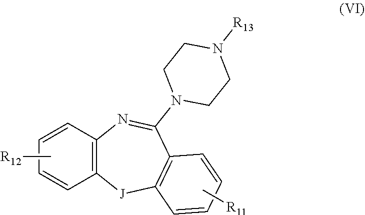
wherein in formula (VI)
R₁₁ and R₁₂ are independently selected from hydrogen, halogen, —OH, (C₁-C₆)alkyl, —CF₃, —O(C₁-C₆)alkyl, —NO₂, —NRₒR_{z}, —SO₂NRₒR_{z}, or —S(C₁-C₆)alkyl;
wherein each Rₒ and R_{z} is independently selected from hydrogen, (C₁-C₆)alkyl;
R₁₃ is hydrogen, allyl, (C₁-C₆)alkyl, preferably (C₁-C₃)alkyl, —O(C₁-C₆)alkyl, or hydroxy(C₁-C₆)alkyl, preferably hydroxy(C₁-C₃)alkyl;
J is S or NH;
or a pharmaceutically acceptable salt, prodrug, enantiomer, diastereomer, racemic mixture, crystalline form, amorphous, unsolved form or solvate of the general formula (I), (II), (III), (IV), (V) or (VI).

3. The method of claim 1, wherein the 5-HT7 receptor antagonist is selected from the group consisting of, Amisulpride, Lurasidone, Vortioxetine, Mianserin, Clozapine or SB-269970.

4. The method of claim 1, wherein the 5-HT7 receptor antagonist is SB-269970.

5. The method of claim 1, wherein the antagonist is a small organic molecule comprising at least two carbon atoms having a molecular weight in the range between 100 and 1000 Dalton.

6. A method for treating or preventing a tauopathy in a subject in need thereof, the method comprising administering to the subject a pharmaceutical composition comprising the 5-HT7 receptor antagonist according to claim 1.

7. The method of claim 1 wherein preventing or treating a tauopathy is achieved by prevention of both the receptor-induced phosphorylation and accumulation of Tau proteins.

8. The method of claim 1, wherein tauopathy is dementia-associated tauopathy.

9. The method of claim 1, wherein tauopathy is selected from the group consisting of Alzheimer's disease, frontotemporal dementia, primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), Huntington disease, glial globular tauopathy, amyotrophic lateral sclerosis (ALS), Parkinson's disease, spinal muscular atrophy (SMA), cerebral amyloid angiopathy (CAA).

10. The method of claim 6, wherein preventing or treating a tauopathy is achieved by prevention of both the receptor-induced phosphorylation and accumulation of Tau proteins.

11. The method of claim 6, wherein tauopathy is dementia-associated tauopathy.

12. The method of claim 6, wherein tauopathy is selected from the group consisting of Alzheimer's disease, frontotemporal dementia, primary age-related tauopathy (PART), chronic traumatic encephalopathy, progressive supranuclear palsy (PSP), corticobasal degeneration, dementia with Lewy Bodies (DLB), frontotemporal dementia and parkinsonism linked to chromosome 17 (FTDP-17), argyrophilic grain disease (AGD), Huntington disease, glial globular tauopathy, amyotrophic lateral sclerosis (ALS), Parkinson's disease, spinal muscular atrophy (SMA), cerebral amyloid angiopathy (CAA).


SEQUENCE LISTING (8 sequences)

SEQ ID NO: 1 - DNA, 19 bp, Artificial sequence
  Target sequence for short hairpins_1
actaccgttgttataggtg

SEQ ID NO: 2 - DNA, 19 bp, Artificial sequence
  Target sequence for short hairpins_2
tcctttatcattatggtgt

SEQ ID NO: 3 - DNA, 20 bp, Artificial sequence
  GAPDH_forward primer
tgcaccaccaactgcttagc

SEQ ID NO: 4 - DNA, 21 bp, Artificial sequence
  GAPDH_reverse primer
ggcatggactgtggtcatgag

SEQ ID NO: 5 - DNA, 26 bp, Artificial sequence
  probe_1
ccctggccaaggtcatccatgacaac

SEQ ID NO: 6 - DNA, 24 bp, Artificial sequence
  WPRE_forward primer
cctggttgctgtctctttatgagg

SEQ ID NO: 7 - DNA, 19 bp, Artificial sequence
  WPRE_reverse primer
tgacaggtggtggcaatgc

SEQ ID NO: 8 - DNA, 25 bp, Artificial sequence
  probe_2
cgttgtcaggcaacgtggcgtggtg